(19) United States
(12) Patent Application Publication
(10) Pub. No.: US 2018/0235470 A1
(43) Pub. Date: Aug. 23, 2018

(54) DEVICES, SYSTEMS AND METHODS FOR THE DETECTION OF ANATOMICAL OR PHYSIOLOGICAL CHARACTERISTICS

(71) Applicant: AUM Cardiovascular, Inc., Northfield, MN (US)

(72) Inventors: Marie JOHNSON, NORTHFIELD, MN (US); Jixing YAO, NORTHFIELD, MN (US); Pranita PATIL, NORTHFIELD, MN (US); Mike LARSON, NORTHFIELD, MN (US)

(21) Appl. No.: 15/507,192

(22) PCT Filed: Aug. 28, 2015
(86) PCT No.: PCT/US2015/047530

§ 371 Date: Feb. 27, 2017

Related U.S. Application Data
(60) Provisional application No. 62/043,052, filed on Aug. 28, 2014.

(51) Int. Cl. A61B 5/00; A61B 5/0205; A61B 7/04; G16H 40/63; G16H 50/20; G16H 10/65

(52) U.S. Cl. CPC A61B 5/0022; A61B 5/0205; A61B 5/02055; A61B 5/6852; A61B 5/686; A61B 5/0816; A61B 7/04; G16H 40/63; G16H 50/20; G16H 10/65; A61B 5/7267

(57) ABSTRACT

Embodiments relate to non-invasive medical devices, systems and methods for the detection of anatomical or physiological conditions. In an embodiment, a handheld coronary artery disease (CAD) detection device is used in a non-invasive manner to determine whether an internal coronary artery blockage, calcification or diffuse disease is present. In an embodiment, a data transfer system comprises a hand-held CAD detection device or other data collection device, and a networked or local computer system for storing and processing the recorded data. Supplemental devices such as a data portal device, hub, or dongle can facilitate data transfer between the data collection device and networked or local computer system.

### RELATED APPLICATION

The present application claims the benefit of U.S. Provisional Application No. 62/043,052 filed Aug. 28, 2014, which is hereby incorporated herein in its entirety by reference.

### TECHNICAL FIELD

Embodiments relate generally to medical devices, and more particularly to non-invasive medical devices, systems and methods for the detection of coronary artery and other diseases.

### BACKGROUND

Cardiovascular disease is the leading cause of death in both men and women in the United States, and is a major cause of death throughout the world. According to a 2006 American Heart Association (AHA) report, approximately 80 million people in the United States have heart disease and in 2005, 864,480 people lost their lives. This accounts for 35.3 percent of all deaths or one of every 2.8 deaths in the United States according to the AHA. Cardiovascular cost the health care system approximately $368.4 billion in 2004, accounting for nearly a third of the trillion dollars spent on health care in the United States each year, again according to the AHA. Patient care accounts for 90% of this cost.

The health care system would benefit tremendously by identification of those individuals at high risk for coronary related attacks. Current evidence shows that established cardiac risk factors, such as certain abnormal levels of blood pressure, blood glucose and cholesterol and a history of smoking, possess a limited ability to estimate cardiac risk. In symptomatic patients with suspected cardiovascular disease, there are a variety of tests available to establish diagnosis. It remains a difficult problem, however, as clinical history and additional information is needed to establish the diagnosis, estimate prognosis and guide appropriate treatment. Coronary angiography is considered the “gold standard” for diagnosis, but it is invasive and costly and is an appropriate initial diagnostic study in only a minority of patients.

Other tests include exercise treadmill test, stress echocardiogram, computed tomography, calcium heart scanning and angiography. Each of these tests is ordered by clinicians after a patient is suspected to have Coronary Artery Disease (CAD). These tests vary in their accuracy with angiogram considered the gold-standard. Exercise electrocardiogram (ECG) testing is the most commonly used test because it is simple and inexpensive. The patient must be able to exercise to at least 85 percent of the predicted maximal heart rate to rule out ischemic heart disease if the test is otherwise negative. For patients who cannot exercise, have baseline ECG abnormalities that could interfere with exercise ECG testing, or in whom the exercise ECG test suggests intermediate risk, a number of alternative noninvasive tests are available including echocardiography with exercise or pharmacologic, radionuclide myocardial perfusion imaging (rMPI), using either planar or photon emission computed tomographic as the imaging method, positron emission tomography (PET) or using coronary calcium scores. Many of these tests are also invasive, time-consuming and expensive, requiring trained personnel and capital equipment.

Moreover, the aforementioned hospital-centric devices are traditionally coupled to a hospital or caregiver network in order to facilitate data transfer and data flow of the recorded data. No built-in network for data transfer and data flow exists for non-hospital-centric devices. As a result, traditional portable devices often are required to contain both sensing and analyzing components, which can be difficult and costly to engineer. Further, field-ready devices often lack the computing power to adequately analyze the recorded data. In other cases, data transfer networks must be created ad-hoc. Therefore, there is a need for improved devices, systems and methods for the detection of coronary artery disease and other diseases.

### SUMMARY

Embodiments of the present application substantially address or meet the aforementioned needs of the industry. In an embodiment, a system comprises a handheld CAD detection device or other data collection device that can measure data relating to an anatomical or physiological feature, and a networked system for storing, securely transferring, and/or processing the recorded data. Further, supplemental devices such as a computer, hub, smartphone, dongle, or other mobile networked device can facilitate data transfer between the data collection device and network-based system.

In embodiments, a handheld detection device can include components for properly placing and positioning the detection device, as well as acting as a stand-alone device which can facilitate data transfer. For example, in various embodiments, a camera, light sensor, temperature sensor, accelerometer, and/or gyroscope can be utilized. In embodiments, the aforementioned components further assist in noise reduction or noise cancellation. In alternative embodiments, an internal detection device can be arranged within a patient, either as an implantable device or as a catheter or other movable device to measure attributes of the patient's anatomy or physiology.

The above summary is not intended to describe each illustrated embodiment or every implementation of the invention. The figures and the detailed description that follow more particularly exemplify these embodiments.

### BRIEF DESCRIPTION OF THE DRAWINGS

Embodiments may be more completely understood in consideration of the following detailed description in connection with the accompanying drawings, in which:

FIG. 1 is a block diagram of a system for the detection of coronary artery disease according to an embodiment.

FIG. 2 is a block diagram of a system for the detection of coronary artery disease according to an embodiment.

FIG. 3 is block diagram of a system for the detection of coronary artery disease according to an embodiment.

FIG. 4 is a flowchart of a method of data transfer using a system for the detection of coronary artery disease according to an embodiment.

FIGS. 5A-5F are flowcharts of portions of methods of data transfer according to an embodiment.

FIG. 6A is a top perspective view of a coronary artery disease (CAD) detection device according to an embodiment.

FIG. 6B is a bottom perspective view of a coronary artery disease (CAD) detection device according to an embodiment.

FIGS. 7A-7C depict catheter-based systems for detection of an anatomical or physiological condition according to embodiments.

FIGS. 7D-7F are data outputs corresponding to anatomical or physiological conditions according to three embodiments.

FIG. 8A is a depiction of a scanning guide according to an embodiment.

FIG. 8B is a depiction of the front cover of the scanning guide of FIG. 8A according to an embodiment.

FIG. 9 is a block diagram of a system for the detection of coronary artery disease including a hub according to an embodiment.

FIG. 10 is a block diagram of a CAD detection device according to an embodiment.

FIGS. 11A and 11B are diagrams of a host computer including the system of FIG. 8 according to an embodiment.

FIGS. 12A-12I depict a data syncing method with a CAD detection device utilizing a dongle according to an embodiment.

FIGS. 13A and 13B are perspective views of a system for the detection of coronary artery disease including a dock according to an embodiment.

FIGS. 14A-14N depict the operation of a CAD detection device with a scanning guide according to an embodiment.

FIGS. 15A-15G depict a number of failsafe user interface displays for a CAD detection device according to an embodiment.

FIG. 16 is a schematic depiction of a neural network according to an embodiment.

While embodiments are amenable to various modifications and alternative forms, specifics thereof have been shown by way of example in the drawings and will be described in detail. It should be understood, however, that the intention is not to be limited with respect to the particular embodiments described. On the contrary, the intention is to cover all modifications, equivalents, and alternatives falling within the spirit and scope of the appended claims.

### DETAILED DESCRIPTION OF THE DRAWINGS

Embodiments relate to non-invasive handheld or implantable medical devices, systems and methods for the detection of coronary artery and other diseases. In an embodiment, a handheld data detection device is used in a non-invasive manner to determine whether an internal coronary artery blockage is present. In other embodiments, the data collection device can be used to diagnose pulmonary disease, carotid disease, renal artery disease, valve disease or other disorders in addition to or instead of coronary artery blockage or disease. In an embodiment, a data transfer system comprises a handheld data collection device or other data collection device, and a networked system for storing and processing the recorded data. The handheld data collection device can measure acoustic and other signals, and these data can be used to assess a likelihood of CAD, pulmonary disease, or other disorders that can be sensed by obtaining such data from the test subject, accurately and efficiently as compared to conventional techniques. In various embodiments, detection devices can detect anatomical aspects (i.e., the physical dimensions or structure of an anatomical feature) or physiological aspects (i.e., how an anatomical feature is performing) or both.

Although many embodiments described herein refer to detection of CAD, it should be understood that in embodiments the devices, systems, and methods described herein could be used to collect data corresponding to a variety of other cardiovascular or pulmonary systems, or indeed any other system in which the collection and transmission of acoustic data can be used in the detection and/or diagnosis of a disease or condition. Such conditions can include, for example, cardiac, pulmonary, valvular, renal, peripheral and/or carotid artery functions. In those embodiments below in which cardiac artery disease is discussed, it should be understood that in alternative embodiments the detection systems, devices, and methods are not limited to the treatment, detection, or diagnosis related to just one particular type of data. These conditions are often not limited to humans, and as such the devices, systems, and methods described herein can likewise apply in veterinary applications.

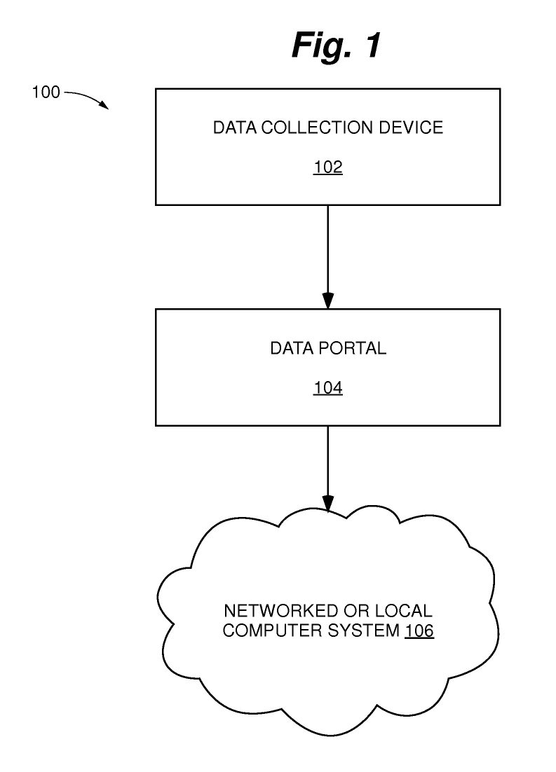

Referring to FIG. 1, a block diagram of a data transfer system 100 is depicted, according to an embodiment. Data transfer system 100 generally comprises a data collection device 102, a data portal device 104, and a networked or local computer system 106, which could include, for example, a remote server system such as those referred to as “cloud-based” systems. In alternative embodiments, other networked systems (either wired, wireless, remote, or local) can be used rather than networked or local computer system 106. Networked or local computer system 106 is only one implementation of a networked computer system that can include storage and processing capabilities.

Data collection device 102 comprises a device for recording, sensing, or otherwise collecting data. In an embodiment, data collection device 102 is configured to collect data from a human or animal patient. In an embodiment, data collection device 102 comprises a CAD detection device, as will be described further below. Data collection device 102 can be configured to be wireless and portable so as to collect data in remote places or places without a traditional hospital infrastructure.

Data portal device 104 comprises a base station or portal configured to interface to at least one data collection device 102. In embodiments, data portal device 104 comprises a desktop computer, laptop computer, smartphone, personal digital assistant (PDA), tablet, watch, wearable electronic device, or other suitable device. In embodiments, data portal device 104 acts as a pass-through to transfer collected data from one or more data collection devices 102 to networked or local computer system 106. In embodiments, data portal device 104 can also consolidate, combine, and/or package data in addition to transferring or broadcasting it.

In one embodiment of a pass-through device, data portal device 104 can be a dedicated pass-through device. Plug-in or battery-operated devices can be positioned in a location proximate to expected testing operations. For example, a hub device such as those commercially available from a multiplicity of companies can be plugged into a wall outlet or battery powered in an examination room where data collection device 102 is to be used. Such pass-through devices can be configured to activate upon wirelessly receiving data from associated devices (such as data collection device 102) and pass on the information to a remote device (such as networked or local computer system 106). In embodiments, data transfer can be accomplished via WiFi networks, cellular networks, mobile data networks, Bluetooth, near-field communications networks, radio frequency networks, wired Ethernet or telephone line networks, or any other transmission medium to networked or local computer system 106.

In embodiments, data collection device 102 and data portal device 104 are operably coupled by a communication network and suitable hardware. For example, both data collection device 102 and data portal device 104 can comprise Universal Serial Bus (USB), Firewire, Bluetooth, serial, EEPROM, WI-FI, or any other appropriate hardware, software, and suitable interfaces. In embodiments, data collection device 102 is configured with minimal data transmission hardware (i.e. with hardware necessary to transmit to data portal device 104), so that data collection device 102 can be more inexpensively and efficiently produced. In such embodiments, data portal device 104 can be configured with additional communication hardware to receive data from data collection device 102 and transmit the data to any number of configurations of networked or local computer system 106. In other embodiments, data collection device 102 can include some data storage capability, enabling the storage of a desired number of previous test results, software, or other information.

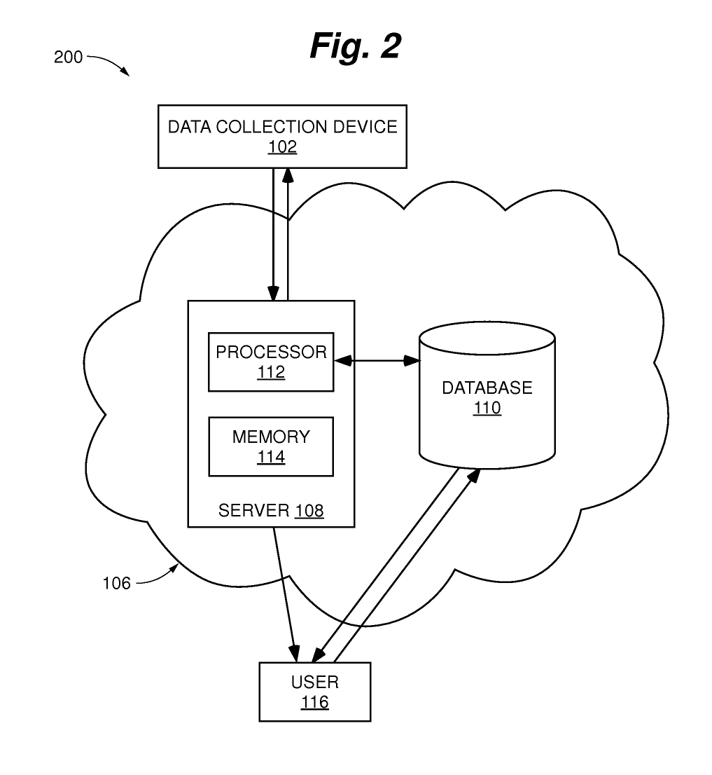

Networked or local computer system 106 comprises data storage and at least one processing engine, as will be described further with respect to FIG. 2. In embodiments, networked or local computer system 106 is configured to store and process data received from data collection device 102 via data portal device 104, and in some embodiments networked or local computer system 106 is configured to store, process and/or aggregate data received from a plurality of data collection devices 102 via one or more data portal devices 104. In embodiments, networked or local computer system 106 is configured to produce a report, analysis, diagnosis, or other output based on the received data. In embodiments, networked or local computer system 106 therefore comprises an analytics engine. In embodiments, these reporting, analysis, diagnostic, or other processing, storage, or output functions can be shared between networked or local computer system 106 and the other components and devices shown in FIG. 2.

In another embodiment, referring to FIG. 2, a block diagram of a data transfer system 200 is depicted. In the embodiment depicted, data collection device 102 is configured to interface directly to networked or local computer system 106. Data transfer system 200 therefore comprises data collection device 102 and networked or local computer system 106. FIG. 2 also depicts, optionally, user 116.

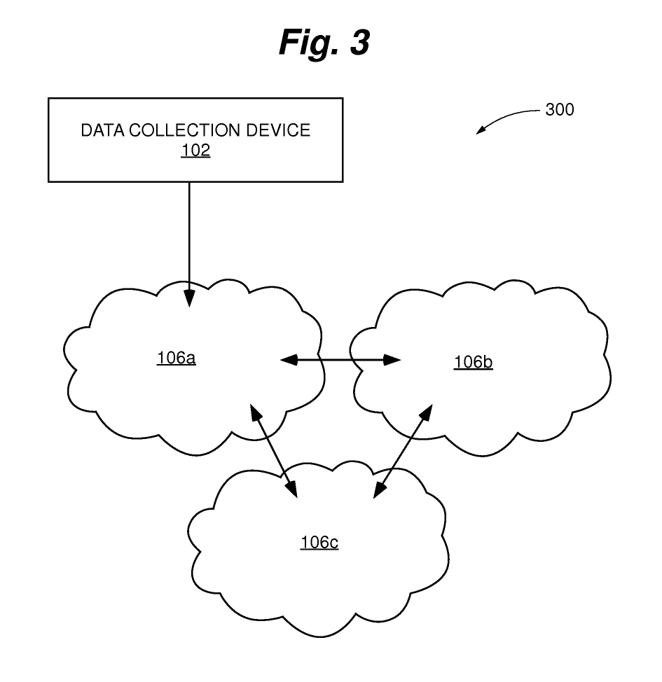

In an embodiment, networked or local computer system 106 generally comprises server 108 and database 110. Networked or local computer system 106 embodies the computation, software, data access, and storage services, and the hardware and devices to implement them to users over a network. The components of networked or local computer system 106 can be located in a singular “cloud” or network, or spread among many devices or networks, as depicted in FIG. 3. End-user knowledge of the physical location and configuration of components of networked or local computer system 106 is not required.

Server 108 generally includes processor 112 and memory 114. Processor 112 can be any programmable device that accepts digital data as input, is configured to process the input according to instructions or algorithms, and provides results as outputs. In an embodiment, processor 112 can be a central processing unit (CPU) or a plurality of processors configured to carry out the instructions of a computer program. Processor 112 is therefore configured to perform basic arithmetical, logical, and input/output operations. In the embodiment shown in FIG. 2, processor 112 can also transmit information to data collection device 102. For example, in embodiments processor 112 can transmit processed results to data collection device 102 for storage therein.

Memory 114 can comprise volatile or non-volatile memory as required by the coupled processor 112 to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the claims.

User 116 can receive information generated by the devices, systems, and methods described above that make up system 200. For example, in embodiments it may be desirable to send information to a health care professional, patient, or other user for access either at or after the time of use of data collection device 102. User 116 can be, for example, an insurance provider, health care provider, patient, or other individual who has access to the test results, in embodiments. User 116 can receive results from data collection device 102 via server 108 at the conclusion of the use of data collection device 102. Alternatively or additionally, as depicted by the arrows connecting user 116 and database 110, user 116 can access results from prior uses of data collection device 102 that have already been sent to database 110. In some circumstances, it may be desirable to compare the results of a recently-measured dataset from data collection device 102 with one or more earlier results, in which case user 116 can receive data from both server 108 and database 110 for comparison or analysis.

As depicted in FIG. 2, server 108 interfaces with database 110 via processor 112. Specifically, processor 112 can execute database-specific calls to store and retrieve data from database 110. Database 110 can comprise any organized collection of data. In embodiments, database 110 can comprise simple non-volatile memory as part of a computer. In embodiments, database 110 can comprise one or more database management systems.

As used herein, the term “processor” can refer to any suitable programmable device that accepts analog or digital data as input, is configured to process the input according to instructions or algorithms, and provides results as outputs. In an embodiment, the processor can be a central processing unit (CPU) configured to carry out the instructions of a computer program. In other embodiments, the processor can be an Advanced RISC (Reduced Instruction Set Computing) Machine (ARM) processor or other embedded microprocessor. In other embodiments, the processor comprises a multi-processor cluster. The processor is therefore configured to perform at least basic selected arithmetical, logical, and input/output operations.

Memory can comprise volatile or non-volatile memory as required by the coupled processor to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. The foregoing examples in no way limit the type of memory that can be used, as these embodiments are given only by way of example and are not intended to limit subject matter hereof. In other embodiments, the memory comprises a plurality of memories or types of memory.

As shown in FIG. 2, database 110 can be discrete from server 108. In another embodiment, database 110 is a part of server 108. In other embodiments, referring to FIG. 3, database 108 can be accessed as part of a separate networked or local computer system 106. Components of networked or local computer system 106 can therefore be spread among multiple networked or local computer systems 106. For example, and referring to FIG. 3 and system 300, server 108 can be spread among many networked or local computer systems 106a-106c. This type of distributed computing is a type of “layered processing.” Database 110 can reside on a first networked or local computer system 106a, while processor 112 and memory 114 reside on a second cloud-based processing engine 106b. Or, processor 112 can reside on a first networked or local computer system 106a, memory 114 can reside on a second networked or local computer system 106b, and database 110 can reside on a third networked or local computer system 106c. Any number of permutations where components are spread among a plurality of networked or local computer systems are considered.

Likewise, system 300 as depicted in FIG. 3 can also comprise a plurality of servers 108 and databases 110. Data collection device 102 can be configured to access a first networked or local computer system 106a, and the processing, storage, and presentation of data can be configured to be spread among second and third networked or local computer systems 106b and 106c, respectively, for example. In order to maintain patient privacy and/or proprietary analyses of patient information, such information can be selectively shared between networked or local computer systems 106a-106c. In this way, data can be compartmentalized, which promotes data security, high processing speed, and can increase available storage. In some embodiments, for example, data from data collection device 102 can be partially analyzed in each of networked or local computer systems 106a-106c, such that the full analysis is not performed in any one place. Furthermore, by storing information in multiple locations, private information relating to the health of those tested can be anonymized.

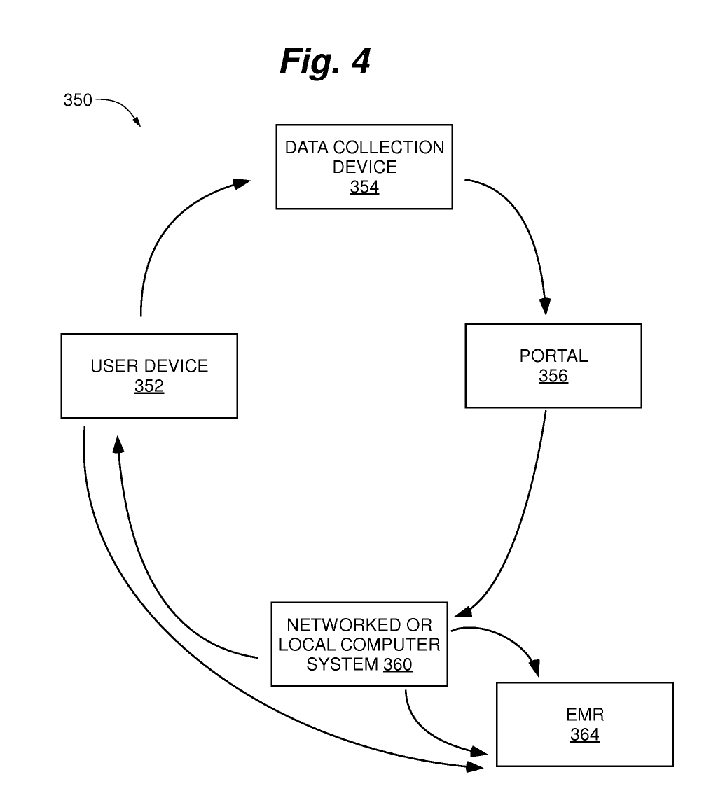

Referring to FIG. 4, a flow diagram for a method of data transfer using a system 350 for the detection of coronary artery disease is depicted, according to an embodiment. In embodiments, data collected by system 350 can be transmitted substantially at the same time that the data is collected. That is, in embodiments, system 350 can upload test data during the test, or within a few minutes after test completion, or upon return to a charging station or docking station. In other embodiments, such as those in which multiple tests are to be performed and analyzed at the same time, test data can be stored until all the data that is desired to be uploaded together has been acquired.

In an embodiment, user device 352 can interface to data collection device 354. In an embodiment, data collection device 354 is substantially similar to data collection device 102. User device 352 can operate data collection device 354 to collect data as described herein. In an embodiment, data collection device 354 comprises a CAD detection device, as will be described further below. Data collection device 354 is configured to interface to portal 356. Portal 356 is configured to operably couple data collection device 354 and networked or local computer system 360. Embodiments of portal 356 will be described further below. Once data is collected with data collection device 354, portal 356 can receive the collected data and transfer it to networked or local computer system 360. In embodiments, data portal 356 can be a dongle attached to a laptop computer or other device, or portal 356 can be a dedicated hub configured to receive and transmit data. In other embodiments, portal 356 can comprise a smartphone, PDA, tablet, laptop computer, desktop computer, watch, other wearable device, or any other suitable networked electronic device. Once the data is received at portal 356 from data collection device 354, portal 356 is configured to transfer the data to networked or local computer system 360.

In an embodiment, networked or local computer system 360 is substantially similar to networked or local computer system 106 as discussed above with respect to FIGS. 1-3. As such, networked or local computer system 360 is configured to store the received data. In an embodiment, networked or local computer system 360 is configured to analyze the data collected by data collection device 354. In an embodiment, networked or local computer system 360 can transfer the raw or analyzed data to Electronic Medical Record (EMR) 364. EMR 364 can then be accessed by the hospital or clinic facility. As indicated by the arrows in FIG. 4, a user (clinician) can also send or upload information directly to EMR 364 via user device 352. In an embodiment, networked or local computer system 360 is configured to store the data collected by data collection device 354.

Once the data from data collection device 354 is analyzed at networked or local computer system 360, networked or local computer system 360 can return a diagnosis or the results of the data analysis to user device 352. In embodiments, the diagnosis or results can be returned in the form of a report, text or email indication, or by other suitable notification. In embodiments (not shown), networked or local computer system 360 can return the diagnosis or results to data collection device 354 or portal 356.

Systems 100, 200, 300, and 350 as depicted in FIGS. 1-4 are depicted by way of example and are not intended to be limiting. Embodiments of networked or local computer systems 106 or components of networked or local computer systems 106 can reside in-house or on a third-party software-as-a-service (SAAS) provider.

In other embodiments, data collection device 102 can be configured to transmit and receive data directly to/from a phone, tablet, laptop, PDA, wearable electronic device (e.g., smartwatch) or other portable electronic device. In embodiments, data collection device 102 is configured with low-power Bluetooth to reduce battery consumption. In other embodiments, data collection device 102 is configured to interface to a dock in a wired or wireless configuration (for example, with low-power Bluetooth). In still other embodiments, data collection device 102 is configured to interface to an intermediate piece of networked hardware, such as a hub. In such embodiments, data collection device 102 is configured to interface to a commercially available data hub configured to push data to networked or local computer system 106. Likewise, data collection device 102 is configured to receive data or commands from a data hub. Other hubs can be used in other embodiments, such as a multipurpose hub that provides data transmission as well as physical docking, calibration, diagnostic testing, battery charging, and other functions.

In other embodiments, data collection device 102 can be configured to stream collected data to a host computer, networked or local computer system, or other electronic device. In such embodiments, the collected data is only temporarily and incompletely stored on data collection device 102, and fully assembled on the device receiving the streamed data, though this need not be the case in all embodiments. In embodiments, a mobile application on, for example, a smartphone, can be configured to receive the streamed data. In embodiments, data collection device 102 can comprise cellular data hardware (4G, LTE, etc.) in order to wirelessly transmit the collected data. In embodiments, data collection device 102 can include its own wireless data plan with a cellular data service provider. In such embodiments, results of data analysis or diagnosis (after transmission of the collected data), as will be described, can be returned or relayed back to data collection device 102, which can present the results on a display. In an embodiment, the results comprise a text-based or color-based indication of a disease diagnosis.

Referring again to FIG. 1, in embodiments of networked or local computer system 106 configured to generate a report, the report can be relayed back to data portal device 104. In other embodiments, the report can be relayed to or made accessible by any other computing device or directly to a user as depicted, for example, in FIG. 2. In still other embodiments, the report can be relayed to a user or users via email or other electronic communication. In embodiments, networked or local computer system 106 is configured such that the generated report remains on networked or local computer system 106.

According to embodiments, a user can access a setup file on data portal device 104 via download or other initial data transfer. In embodiments, to download the setup.exe file, a special code must be entered. In an embodiment, the code is supplied by upon purchase of a system or device, or based on another transaction. In other embodiments, other verification methods can be utilized.

In embodiments, a registration process allows users to create a unique account with username, password, hospital/clinic name, email address and alternate email address to receive reports. Embodiments of the registration process also request user preferences for receiving reports, including file type, such as PDF, WORD, or EXCEL. In embodiments, user preferences include: receive a report by using primary email address, alternate email address or both.

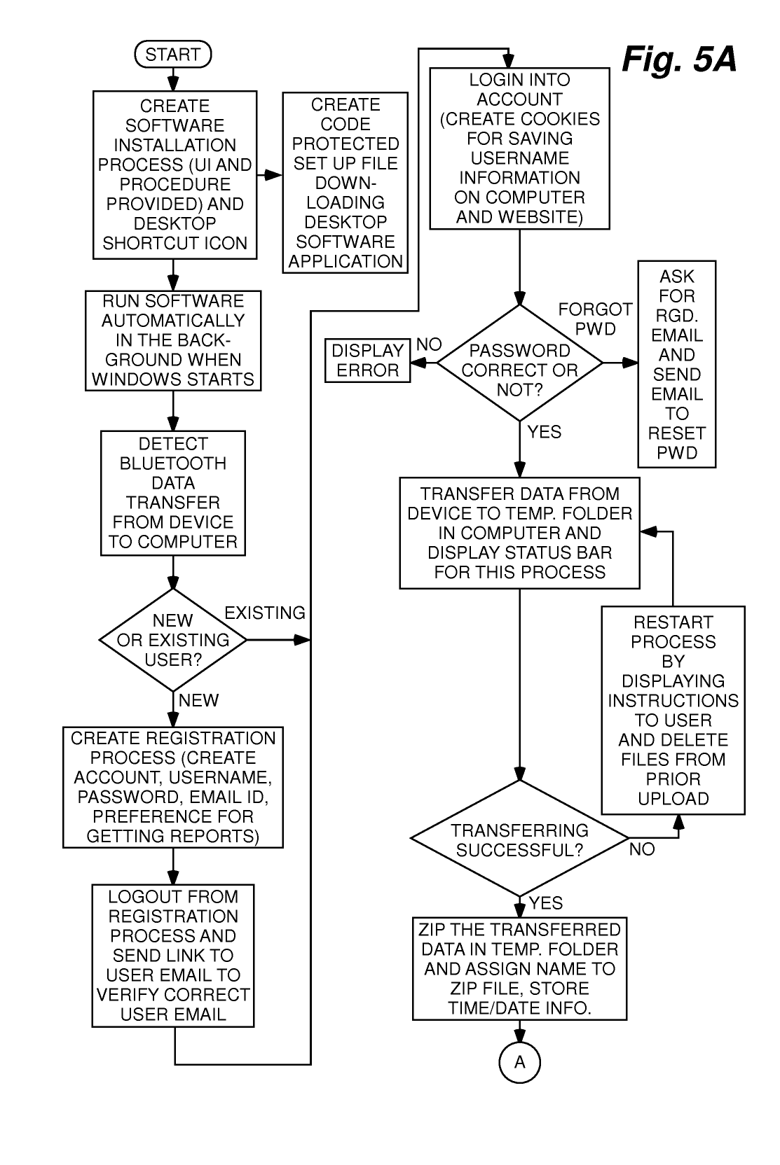

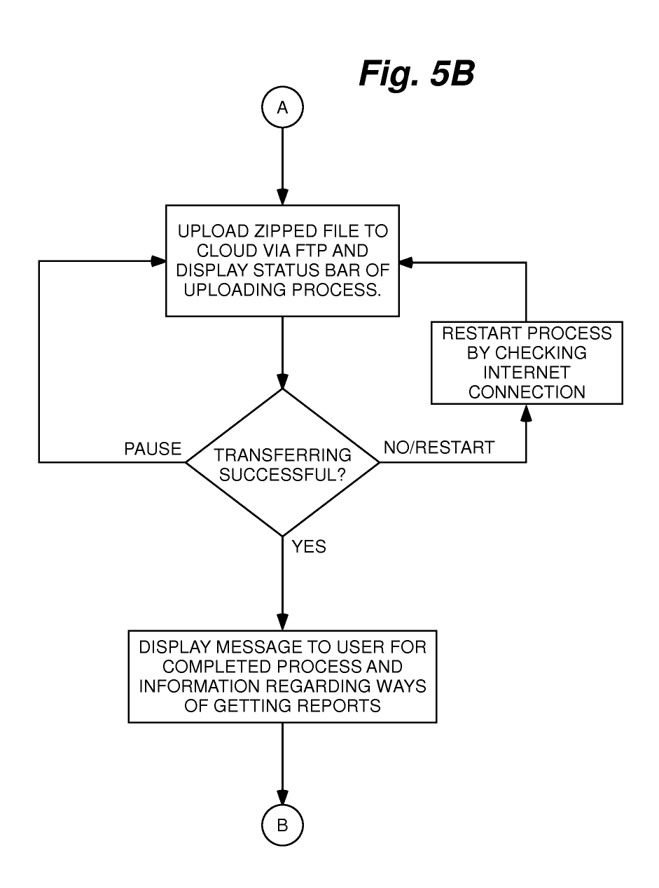

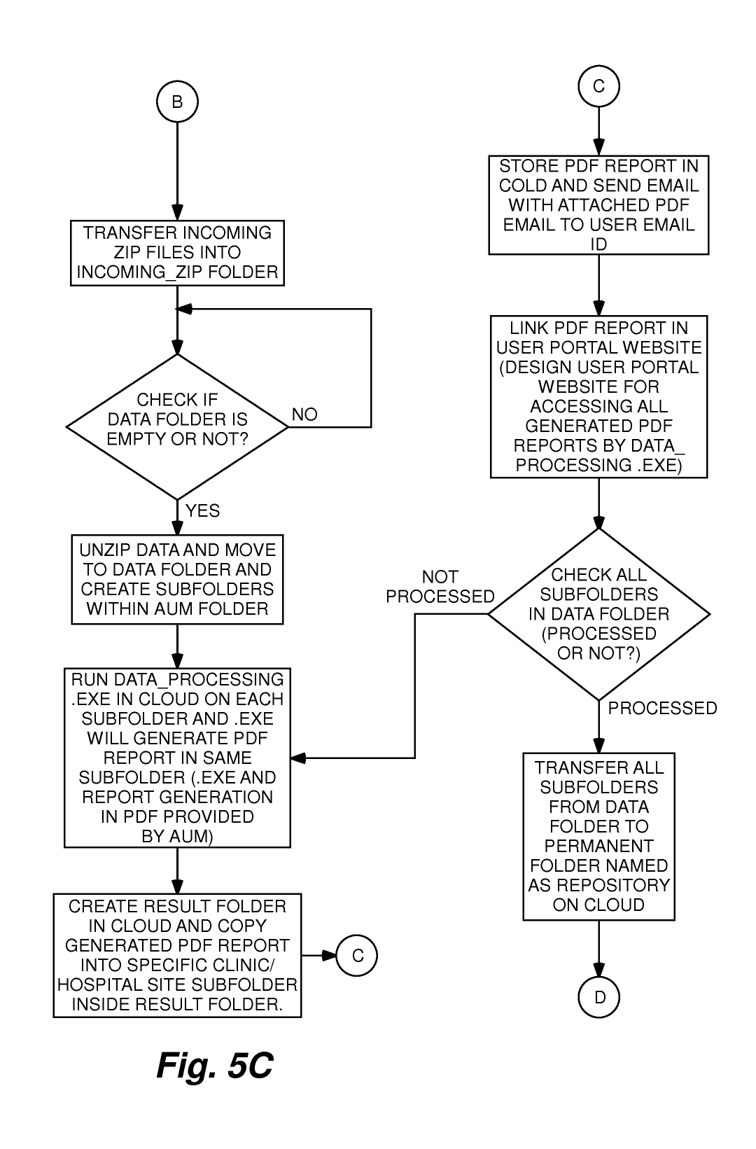

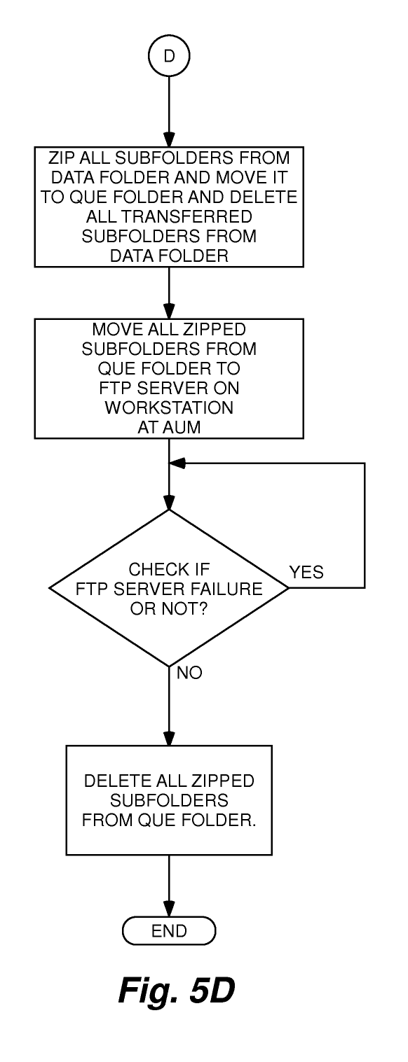

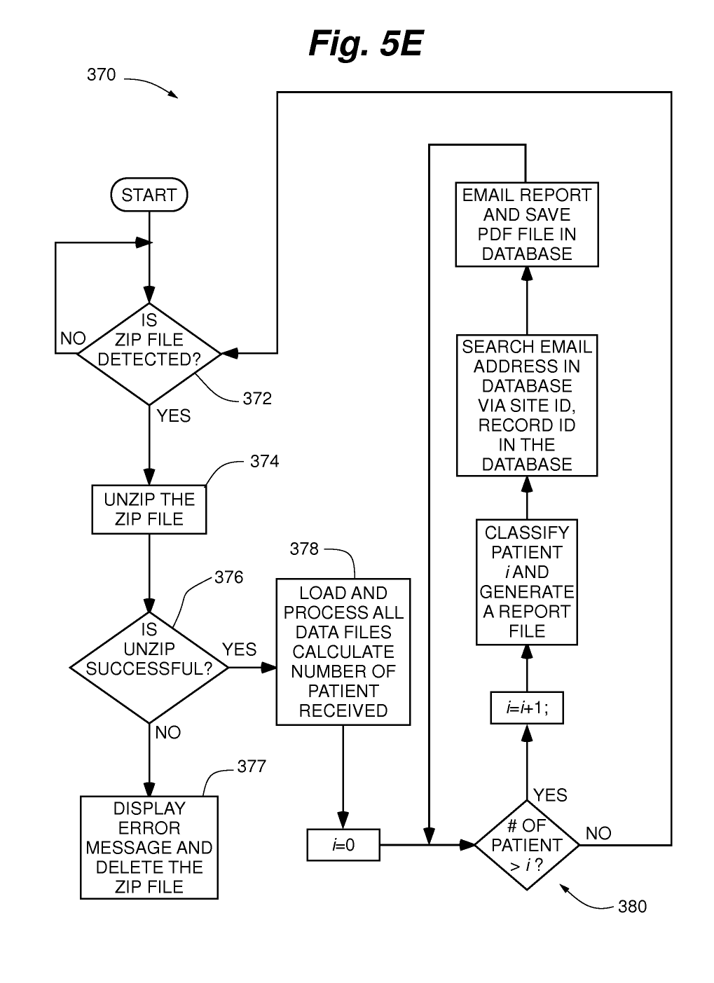

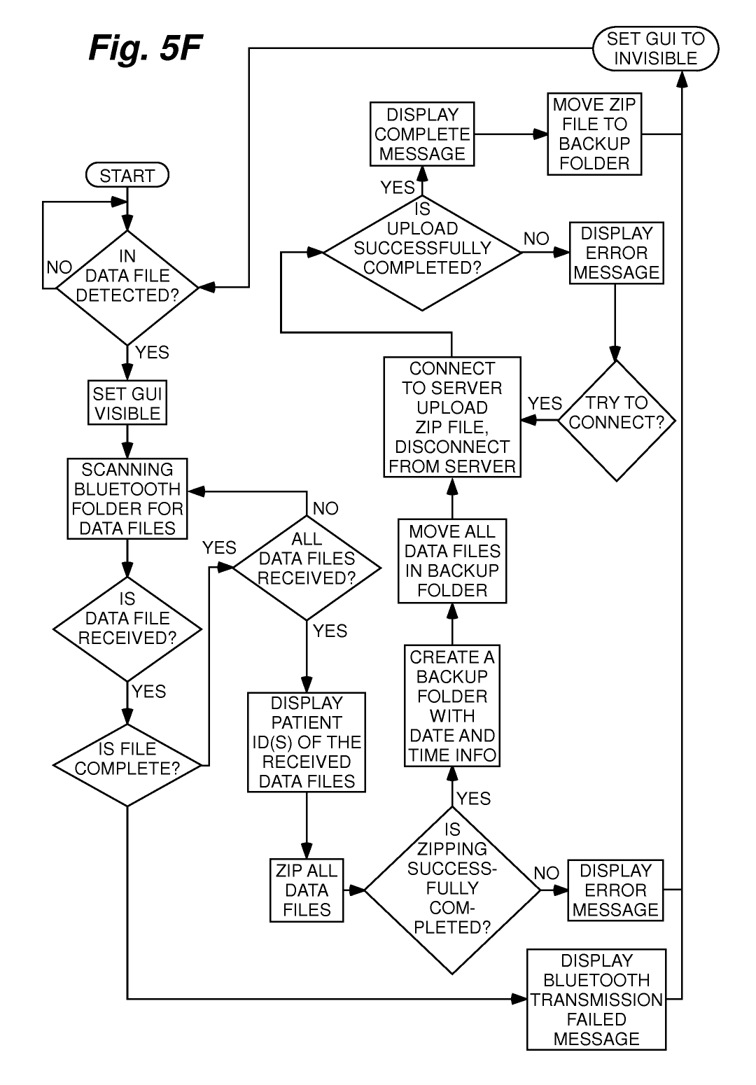

Embodiments of the registration process can be completed on a data portal device 104 software application. Registration information can be stored on a database, such as database 110 or other database. After completing the registration process, logout from the registration process, and verification of the user email address, a link will be sent to that email address which will direct user to login window of host computer software application. After completing registration, embodiments can verify an email address for the user. If the user tries to log in without verifying an email address, then the user will see an error message indicating a need to verify an email address. For each registered user, the database can include the following information: user name, password, hospital/clinic name, email address, billing information, all reports associated to the user, and the date/time when data was uploaded by the user (date/time information stored as part of a zipping process, as will be described with respect to FIGS. 5A-5F).

In embodiments, a user can log in to access his or her account or begin a data sync. In embodiments, the login can be originated from data portal device 104. In such embodiments, data portal device 104 software application(s) can provide direction on how to start the syncing process.

In embodiments, from a user-portal, such as a website interface to networked or local computer system 106, the user can search database 110. In embodiments, searching can be by patient ID or by a date range in which data was uploaded by user. The user can print, download, and/or view reports from the website. In an embodiment, users who have uploaded files onto networked or local computer system 106 can only see documents uploaded by that user. In the website/portal, the user can also change account setting such as email address and password. In embodiments, database 110 is secured so that the user cannot edit any information on database 110.

Further, the user-portal or website interface to networked or local computer system 106 can be configured to present not only reports, but also raw, aggregated, summed, analyzed, or other data to the user or medical professional. In embodiments, metadata for the data can be utilized to present data on different geographic regions, particular hospitals or clinics, or specific age ranges. Such display can facilitate the identification or determination of possible patterns for specific criteria. In embodiments, networked or local computer system 106 comprises different algorithms depending on the type of disease to be diagnosed. For example, data can be analyzed according to different algorithms for coronary artery disease, other vascular disease, hypertension, erectile dysfunction, carotid evaluation, efficacy of tube or other medical device placement, transplant efficacy, and so on.

Upon initiation of data transfer, embodiments of systems are configured for data management within data portal device 104. In an embodiment, a pop up window is presented for an account login procedure if the user is new. In another embodiment, a pop up window is presented for a login for an existing user. In embodiments of the pop up window, username and password tabs are shown and “forgot password option” are presented at the bottom of the same window. If the user has forgotten his password, then a forgotten password option directs the user to the password reset webpage, which asks for the user's registered email ID or username. The password-reset link is sent to the registered email. If the email ID or username is not registered, a link directs the user to a registration process (as will be described further below). In an embodiment, cookies for saving the username will be created on the host computer software as well as on embodiments of the website. A checkbox for remembering the username can be provided on the same window.

A unique patient ID number can be associated with each data file. At times the number of data files and size of data files will require the data file to be compressed prior to sending to the networked or local computer system. There can be any number of files per data set. Data files can store myriad types and kinds of data. In an embodiment, a data file for a particular user and scan can include examination or patient-specific information, such as examination time, examination date, patient height, patient weight, location, etc. In embodiments, such information can be typed in manually via a docking station, as will be described, or through a device interface itself.

In operation, referring generally to FIGS. 5A-5F, the data transfer process can be initiated by data collection device 102. In an embodiment, the data transfer process is initiated via a scan of an RFID tag. In embodiments, the RFID tag can be embedded in a Bluetooth device. In other embodiments, data portal device 104 and/or networked or local computer system 106 can initiate the data transfer process. Embodiments of systems are configured to detect data transfer events and direct all incoming files automatically to a temporary folder. As depicted in FIG. 5A, handling is provided for both new and existing users. Further, in embodiments, handling is further provided for account login and access with password.

Embodiments are configured to display the status of the downloading process from data collection device 102 to data portal device 104 on a pop up window. If upload fails, the data upload error window is presented and all previously transferred are deleted and uploading is restarted. In an embodiment, the transferred data is compressed from the aforementioned temporary folder. Further, in embodiments, date and time information associated with the time the data was syncing, can be determined using the user device's computer clock and are stored in the ZIP file.

Referring specifically to FIG. 5B, embodiments further provide for the management of data transfer from data portal device 104 to networked or local computer system 106. In an embodiment, compressed data is transferred from data portal device 104 to networked or local computer system 106 via FTP. No data will be stored on data portal device 104. In other embodiments, temporary or zipped data can remain on data portal device 104, if desired.

In an embodiment, a status of the uploading process can be provided. In certain embodiments, the status can be displayed using a software application executed or displayed by data portal device 104. In embodiments, Pause and Restart options for the uploading process are also provided. For example, Pause and Restart options for data transfer from data portal device 104 to networked or local computer system 106 can be provided in the same status window. In an embodiment, a status bar for downloading data from the data collection device 102 to data portal device 104 and for uploading data from data portal device 104 to networked or local computer system 106 can be shown in the same pop up window. In embodiments, a display window is provided that shows information with directions for searching and downloading reports by, for example, access via email or access via a website. Further embodiments provide display windows and access for troubleshooting if, for example, a report sent via email is routed to the user's spam folder.

Referring to FIG. 5C, embodiments further provide for the management of operations within networked or local computer system 106.

In an embodiment, a data processing program can be executed on, for example, cloud computing service or in a virtual machine in networked or local computer system 106. Each subfolder can be processed separately and a report can be generated by the data processing program in each subfolder. As depicted in FIG. 5C, dataprocessing.exe can be executed. In embodiments, a PDF file is output. In other embodiments, other file formats or series of file formats are output.

Once generated, the PDF file or other output can be detected when the output is created in the subfolder. The output file can be copied to a result folder. In embodiments, different subfolders can be utilized for each hospital or clinic site. Subfolders can be named according to suitable naming convention. Further, in embodiments, when the output file is detected, it can be attached to an email (as will be described further below).

After the output file is created, all the processed subfolders can be transferred to any number of data storage methods and deleted from the DATA folder.

In an embodiment, the generated output report can be emailed to the user. For example, one of the following options can be utilized. First, a .NET application can utilize a mail server. The SMTP info can be provided by any of the parties involved in the data transmission transaction. In another option, an email integrated into the cloud can be utilized. For example, along with the data output attachment, an email body can contain additional information such as patient ID, Final result associated with patient ID, and hospital name which can be retrieved from the same PDF report filename and/or database 110. In an embodiment, the email subject line can be: Site ID, Patient ID “Data Collection Results”. In embodiments, “Data Collection Results” can be specific to to the particular data collection device. Other email templates are also considered.

Referring to FIG. 5D, in an embodiment, unzipped and processed data files and data subfolders can be transferred from the DATA folder to another permanent folder. For example, the secondary permanent folder can be named Repository, and be stored in networked or local computer system 106, and more particularly, in database 110. For backup purposes, the processed subfolders can be zipped along with data files inside that subfolder in the DATA folder, and move the ZIP files to a permanent folder (Folder Name: QUE) on an FTP server or on a physical disk (such as a USB flashdrive). In an embodiment, the QUE folder is created to store the data files in the event the FTP server fails. In an embodiment, an email can be transmitted to relevant parties regarding any failure of the FTP server and all uploaded data subfolders can be automatically deleted from the DATA folder. In embodiments, ZIP files can be moved from the QUE folder to an FTP server automatically every second/minute/hour/day/week. After uploading the ZIP files to the FTP server successfully, the uploaded ZIP files can be automatically removed from QUE folder on networked or local computer system 106. If there are new ZIP files moved into QUE folder during the on-going upload process, these new files can wait for next uploading process and are not be deleted during the on-going upload process.

Referring to FIG. 5E, a process 370 for operating a data portal device (e.g., data portal device 104) is shown. In embodiments, a data portal device or hub can be configured to receive, analyze, and send data relating to multiple tests simultaneously.

A data portal device (e.g., data portal device 104) can be positioned in a location where it can receive data from a data collection device (e.g., data collection device 102). When a test is run, the data collection device can upload a file such as a ZIP file containing test results. These test results can include, among other things, a scan 1D number corresponding to a particular patient, acoustic test results, or other information related to the scan. If a ZIP file is detected (372), the data portal device can proceed to unzip the ZIP file (374), in embodiments. In alternative embodiments, the ZIP file can be transmitted to a remote server (e.g., networked or local computer system(s) 106) to be unzipped.

A check can be performed to determine whether the unzipping process (374) was successful (376). If not, an error message can be displayed and the ZIP file can be deleted (377). If, however, the unzipping process (374) was successful, the unzipped DATA files can be loaded and processed (378).

In some settings, a ZIP file contains data relating to more than one patient. In loop 380, the results from the data files are analyzed sequentially to generate emailed reports relating to each patient. In alternative embodiments, reports can be generated in other formats or can be sent by other mechanisms than PDF, or delivered by alternative mechanisms instead of email. In some embodiments, the components of loop 380 can be carried out at various locations, such as spreading the analysis to generate each report amongst various networked or local computer systems (e.g., networked or local computer systems 106a-106c as shown in FIG. 3).

Referring to FIG. 5F, a method for preparing a ZIP file containing scan data is depicted. The method shown in FIG. 5F can be conducted at a data collection device such as data collection device 102. When a DATA file is detected (i.e., when a scan is successfully completed), the GUI becomes visible. The GUI can become visible by activating a display on the data collection device, for example. The GUI can be implemented on any data portal device or even the device itself. In some embodiments, the GUI can be located on a personal computer, and a program running the background provides a window pop up to show the desired information on screen. In other embodiments, the GUI can be located at a data portal device or hub rather than the data collection device.

In the embodiment shown in FIG. 5F, a Bluetooth connection is used to transfer files. Once the GUI is visible, the Bluetooth folder is scanned for DATA files. If at least one DATA file has been received, and the file is complete, then the patient IDs corresponding to those DATA files can be displayed. The DATA files can be combined into a ZIP file, as well as saved into a BACKUP folder. The ZIP file can then be uploaded to a server. If all of these steps are completed successfully, the GUI can display a message that indicates a successful file transfer, and the ZIP file can be moved to the Backup folder. With the data transfer complete, the GUI can be set to “invisible” again by, for example, shuting off the screen or changing the display to a screen saver.

In the event that there is a failure to create a ZIP file, the process shown in FIG. 5F creates a notification to that effect. For example, as shown in the process in FIG. 5F, failure to see a complete DATA file results in display of a Bluetooth Transmission Failed message on the GUI. If the DATA file is received but zipping is not successfully completed, an ERROR message is displayed on the GUI. In embodiments where the ZIP file is uploaded to one or more servers, corresponding error messages can be produced where there is a failure to successfully upload.

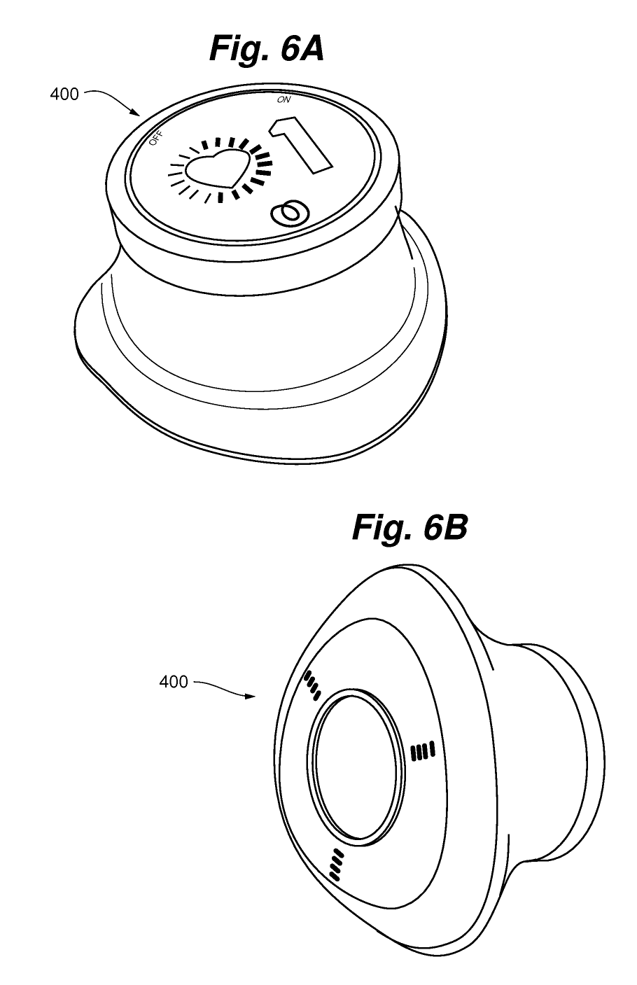

In embodiments, data collection device 102 comprises a device for recording, sensing, or otherwise collecting data. Referring to FIGS. 6A and 6B, in an embodiment, data collection device 102 comprises a handheld coronary artery disease (CAD) detection device 400 that can be used in a non-invasive manner to determine whether an internal coronary artery blockage is present.

As shown in FIGS. 6A and 6B, an amount of battery remaining can be indicated on a display on CAD detection device 400 or data portal device. In some embodiments, this can be displayed as a percentage of battery life remaining, between 0 and 100%. Alternatively, in embodiments where the amount of energy needed to perform each test is at least approximately known, it may be more beneficial to include an indication of the number of tests that can be performed before CAD detection device 400 needs to be charged. In those embodiments, CAD detection device 400 can include both a battery monitor and the ability to run an algorithm to determine how many additional tests the remaining battery charge will be sufficient to accomplish.

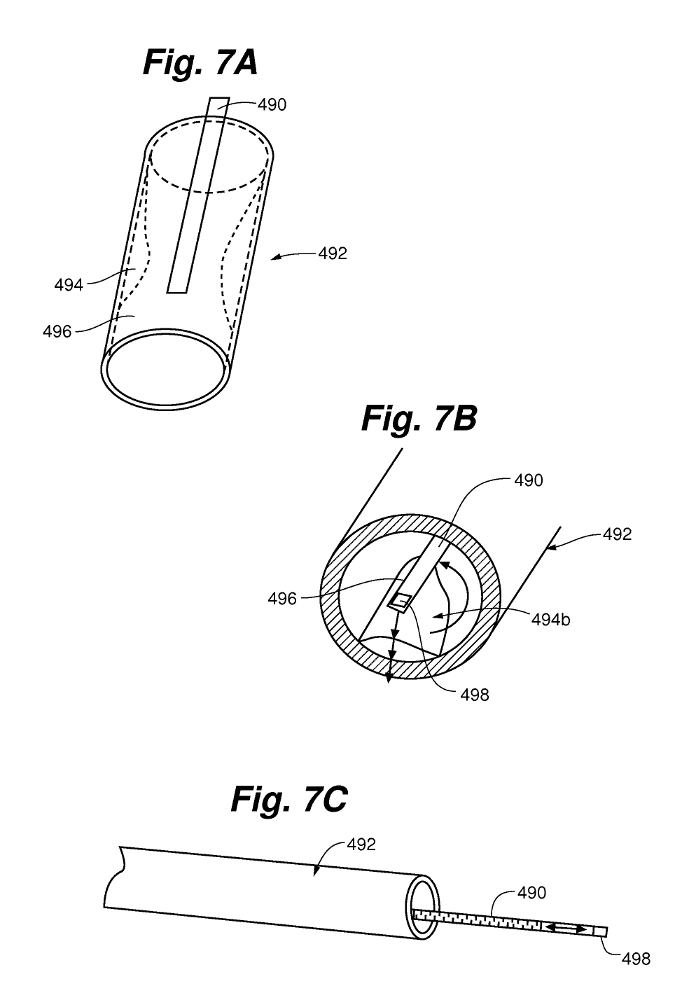

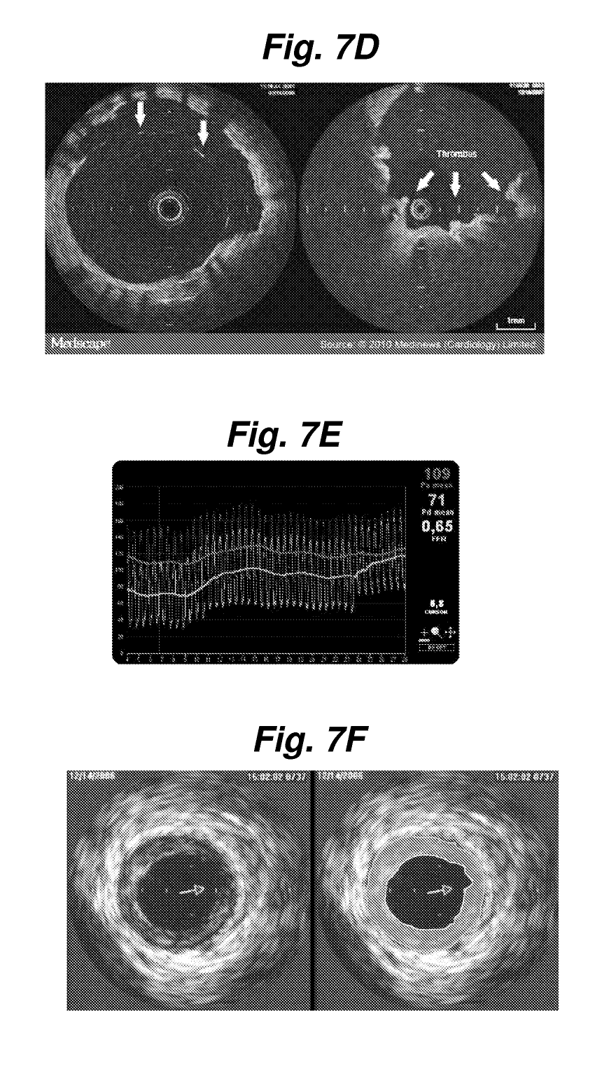

CAD detection device 400 can be used in conjunction with an identification element, such scanning area identification pads or a patient scan sequence guide 500 as shown in FIGS. 7A-7B, to aid in the proper placement of CAD detection device 400 while scanning a patient. Such systems are described in International Publication Nos. WO 2013023041, WO 2011071989, and U.S. Patent Pub. No. 2009/0177107, and U.S. Pat. No. 7,520,860, which are incorporated herein by reference in their entireties.

Referring to FIGS. 7A-7C, implantable or insertable detection systems are depicted. In these embodiments, like those described above with respect to handheld detection systems, various anatomical or physiological features can be sensed and transmitted to remote processors, storage, or devices accessable to users such as caregivers. In the embodiment shown in FIG. 7A, for example, a catheter 490 is introduced into an vessel 492 having a constriction 494. Constriction 494 can cause turbulence at downstream portion 496, which can be detected by sensors (not shown) on or in catheter 490. In some embodiments, vessel 492 can be an artery. Catheter 490 could include any of a variety of other sensors configured to detect various anatomical or physiological characteristics. For example, catheter 490 can be configured to sense flow rates, turbulence and/or pressure, acoustic data, presence or absence of a particular substance, electrical charge or electromagnetic field. These sensors can be placed on the catheter or can extend from the end of the catheter. In embodiments, the sensors can be deployed from the catheter for placement at a specific location in the anatomy of the patient, as shown by the arrows in FIG. 7C, for example.

FIG. 7B depicts a sensor 498 on a catheter 490 configured to measure the thickness of a constriction 4946 on the interior wall of vessel 492. As indicated by the curved arrow, catheter 490 can be rotated such that sensor 498 can detect, via echolocation or some other detection mechanism, a thickness of constriction 494b due to buildup of deposits or other conditions. As depicted by the straight arrows, when using acoustic data such as echolocation, some sound is reflected at each interface. As such, sensor 498 will receive acoustic data reflected at the radially inner portion of constriction 494b, as well as acoustic data reflected at the interface between constriction 494b and the radially interior wall of vessel 492, as well as acoustic data reflected at the radially outer wall of vessel 492. From these data, which sensor 496 can report either via a wired communication pathway through catheter 490 or via a wireless communication mechanism, it can be ascertained whether a buildup or contriction exists and if so what its thickness is. In alternative embodiments, sensor(s) 496 can be routed through catheter 490 and extend longitudinally outward from catheter 490.

FIG. 7C is a perspective view of catheter 490. In embodiments, catheter 490 can provide a lumen along which sensors, fluids, dyes, or other objects or materials can be routed. Catheter 490 can be inserted into vasculature, for example, to reach a location of interest and facilitate sensing of anatomical or physiological indicators, features, or conditions at that location. In alternative embodiments, sensing can take place at a different structure internal to the patient. For example, in embodiments a pacemaker or other permanent or semi-permanent implanted device can be used to sense anatomical or physiological features. In fact, these implantable devices may already have wireless transmission or reception capabilities which can be used to collect anatomical or physiological data for use in the health monitoring systems described herein.

Use of such internally-placed sensors facilitates the use of techniques such as fractional flow reserve (FFR), optical coherence tomography (OCT), and/or intravascular ultrasound (IVUS). Each of these methods can be used to determine if coronary blockages or other conditions are significant enough to warrant an intervention such as a stent or coronary artery bypass operation, for example. These techiques can provide quantitative measurements, which can be used by the practitioner to generate a precise assessment relating to potential blockages or other conditions. FIG. 7D is an output of an IVUS test, accourding to an embodiment. FIG. 7E is an output of an OCT test, according to an embodiment. FIG. 7F is an output of an FFR test, according to an embodiment. FFR, OCT, and IVUS tests can be perfomed by sensors according to the embodiments previously described with respect to FIGS. 7A-7C. These and other tests can be combined concurrently, in embodiments, to provide data from multiple modalities.

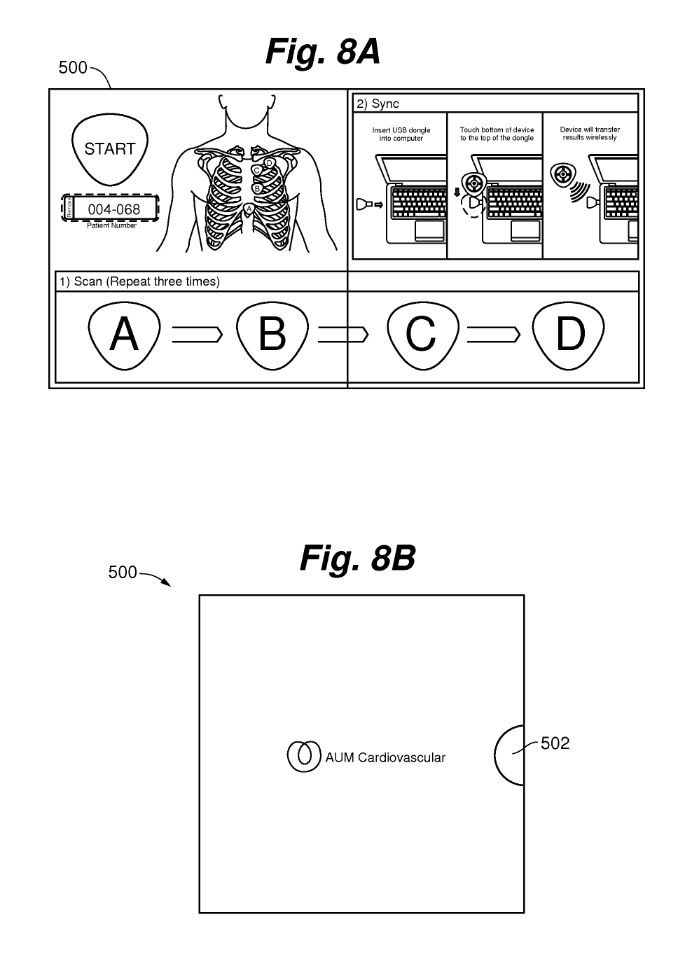

Referring to FIGS. 8A and 8B, sequence guide 500 provides a map for users of a device such as CAD detection device 400 to acquire and send test data. Sequence guide 500 can show an arrangement of positions in which CAD detection device 400 can be placed to acquire desired data. Sequence guide 500 can include multiple RFID chips, NFC chips, or other objects that can store and/or transmit information to the CAD detection device 400. In some embodiments, sequence guide 500 is specific to an individual patient. For example, as shown in FIG. 8A, a “Patient Number” in the sequence guide can be printed over an RFID chip. The “Patient Number” can be known to a limited number of people and therefore data accompanying the Patient Number is de-identified and unknown to any third party that stores or transmits the information. Prior to beginning a test, the Patient Number can be scanned into the CAD detection device 400 from the RFID chip, then transferred to a remote server with the corresponding test data. Using a patient number in this way, in lieu of sending recognizable patient information, can promote anonymous and confidential transmission of information of the test subject.

As shown in FIG. 8A, a sequence of four positions on a human torso are shown. A test-taker can place CAD detection device 400 on the letter corresponding to each test position, then proceed to place the CAD detection device 400 in the appropriate location on the test subject's torso. The letters corresponding to each test position in sequence guide 500 can be positioned above RFID chips, NFC chips, inductive coils, etc., so that the CAD detection device 400 can read that the next data point is about to be taken.

In alternative embodiments, sequence guide 500 could include a variety of other position sequences, and could include more or fewer steps, or could even depict a single location to be scanned. For example, in one embodiment, sequence guide 500 could relate to a test of lungs, rather than a heart, and therefore the positions to be used will be different from those shown on sequence guide 500. In embodiments, sequence guide 500 can include directions for testing of animals, rather than of humans as shown in FIG. 8A. For example, CAD detection device 400 could be used to test heart or lung functions of livestock, racing animals, or pets. In some types of test, it may be beneficial to provide a film or other waterproof barrier on CAD detection device 400 to prevent water or moisture intrusion, or damage. In various embodiments, sequence guide 500 could be an audible or other type of sequence guiding. For example, sequence guide 500 could be implemented on a watch, phone, or other electronic device, in embodiments.

As shown in FIG. 8A, sequence guide 500 gives instructions for use of a Bluetooth dongle attached to a laptop computer via a USB or wireless connection. In alternative embodiments, a remote hub may be used which does not require a USB-connected dongle or a laptop computer. For example, as previously described, in some settings there may be a remote wirelessly-connected hub that remains in place at a testing facility or is otherwise configured for communications with device 400 to receive data from tests. In other embodiments, a smart phone may be configured to receive the data wirelessly, without the use of a USB-connected dongle. In those embodiments, sequence guide 500 can be modified to reflect the physical architecture of the system being used.

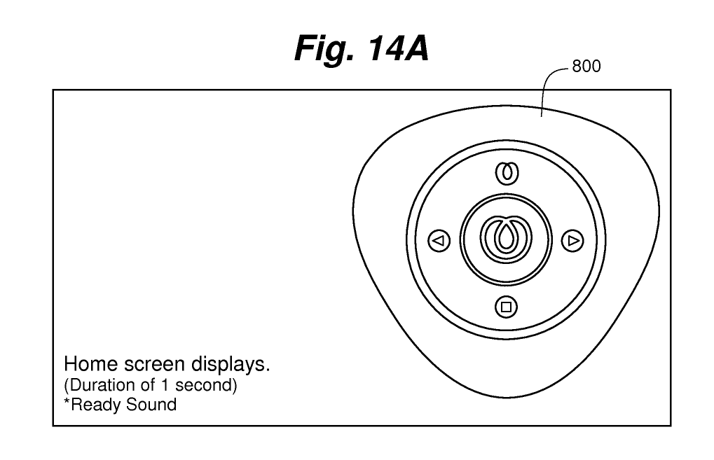

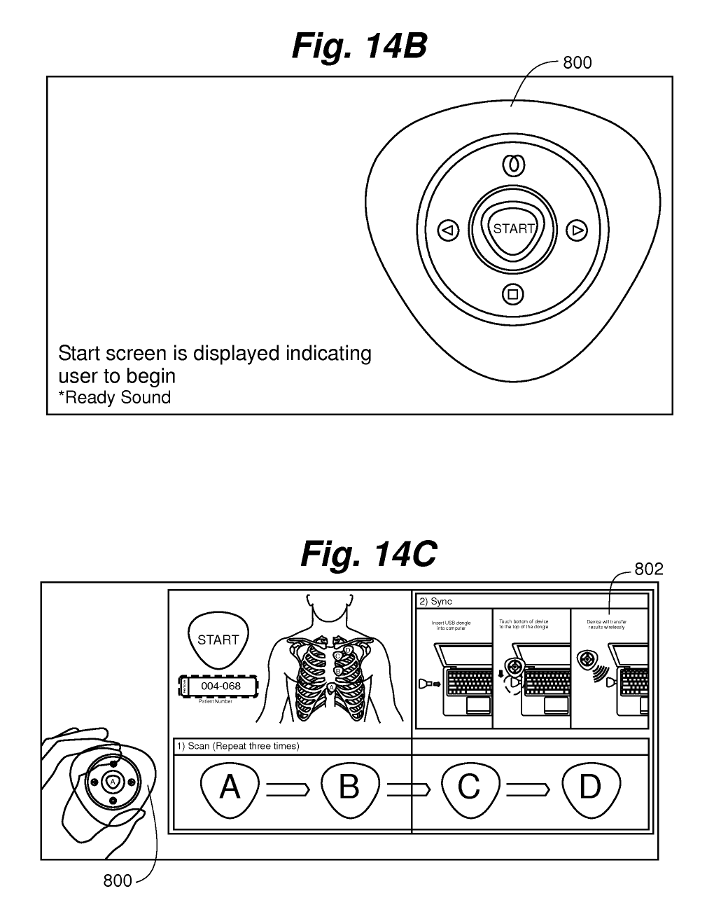

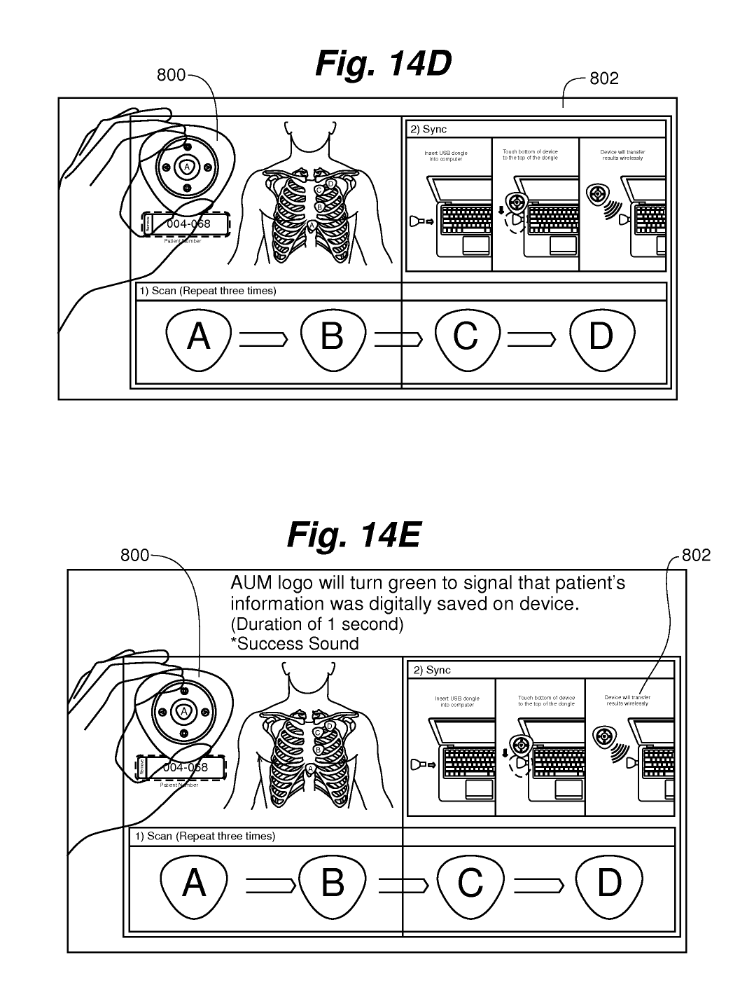

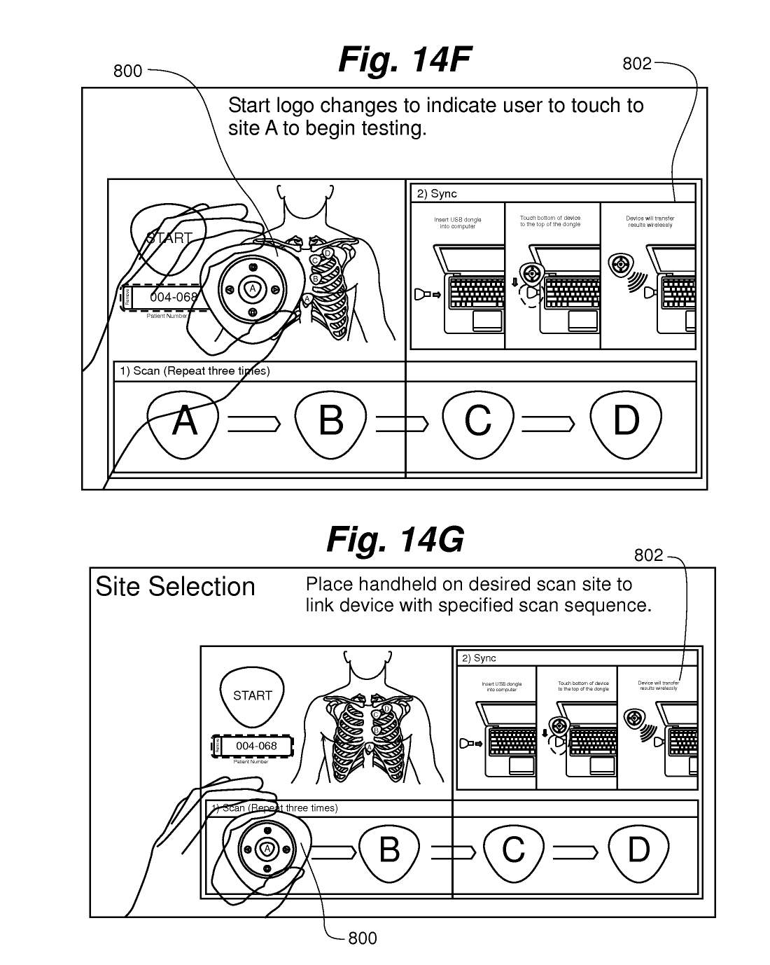

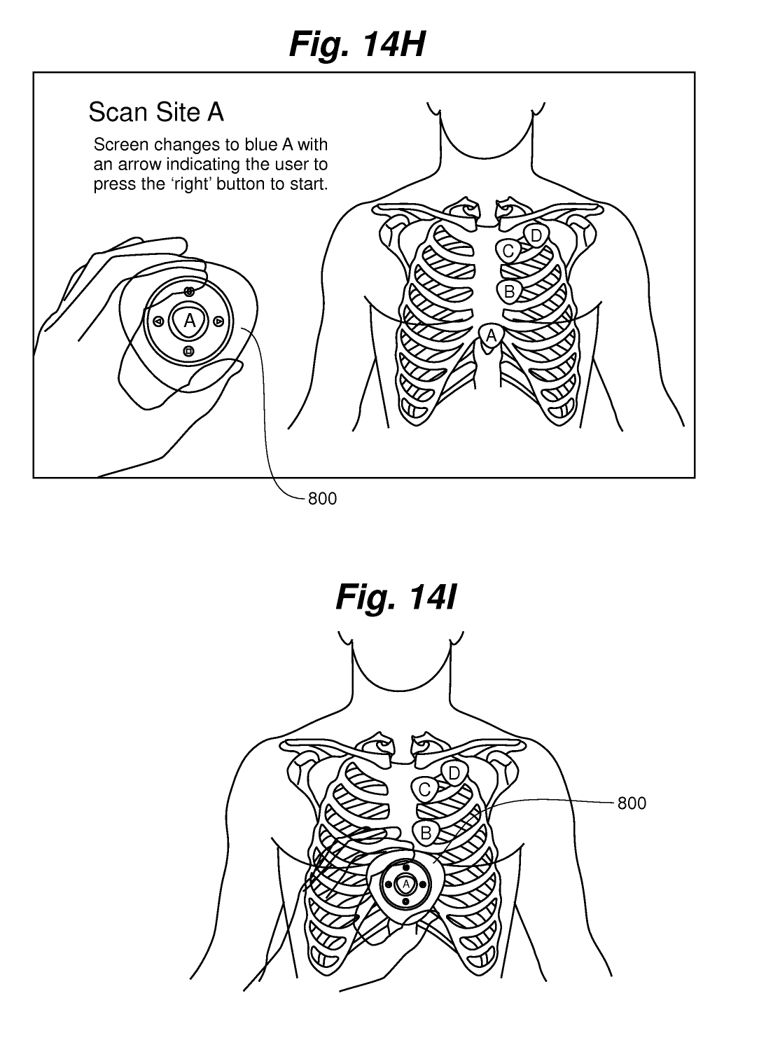

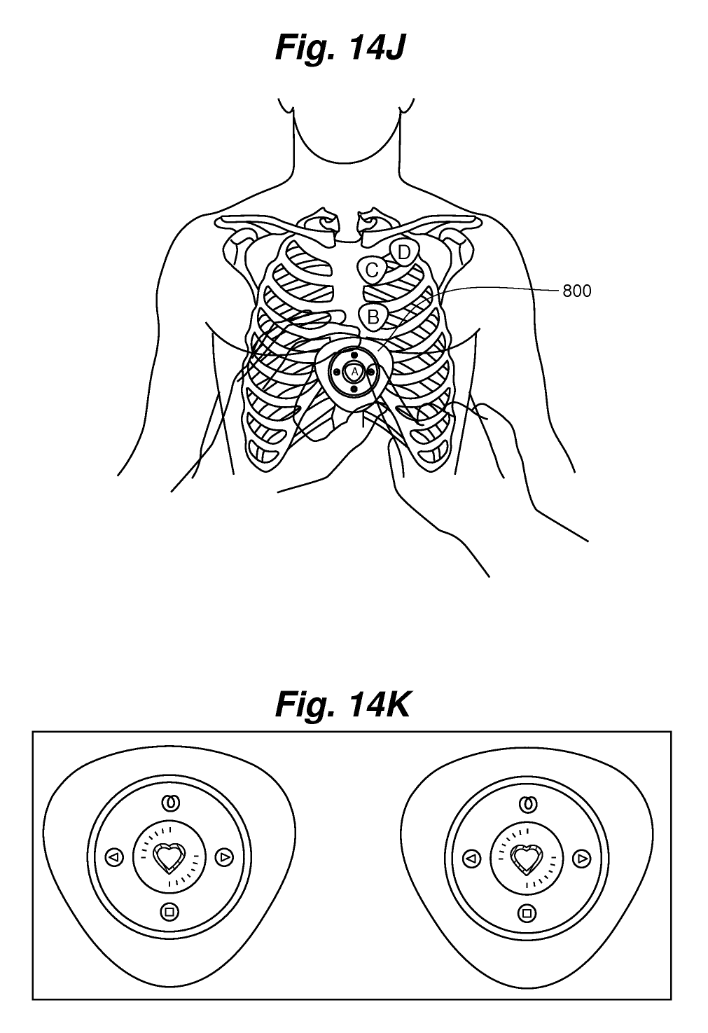

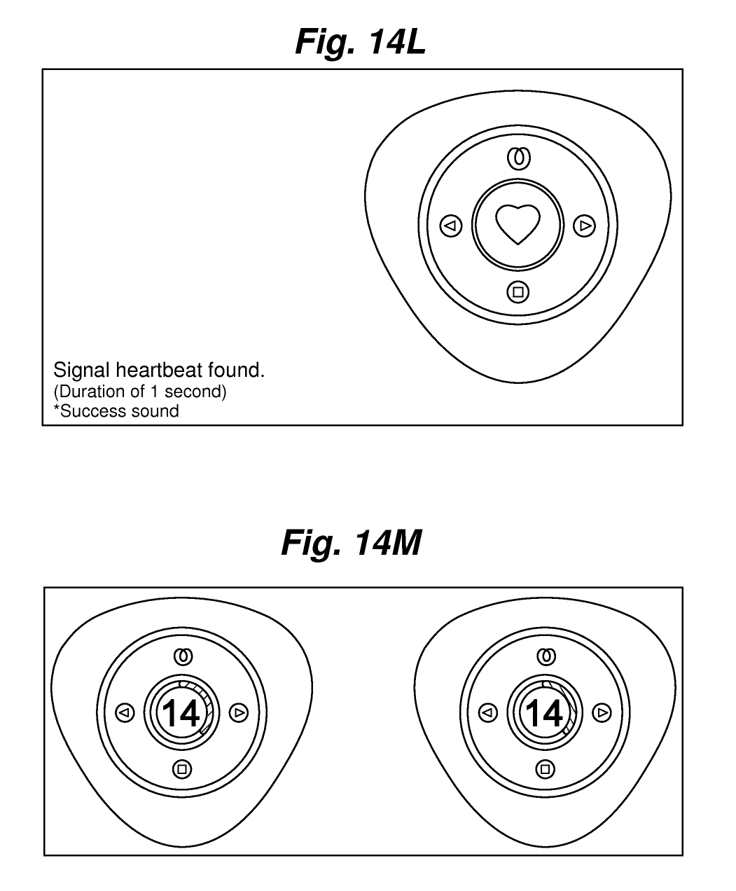

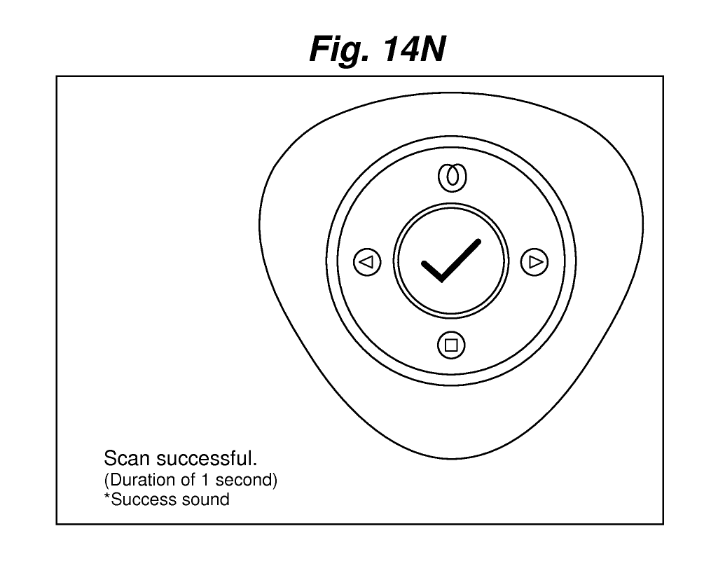

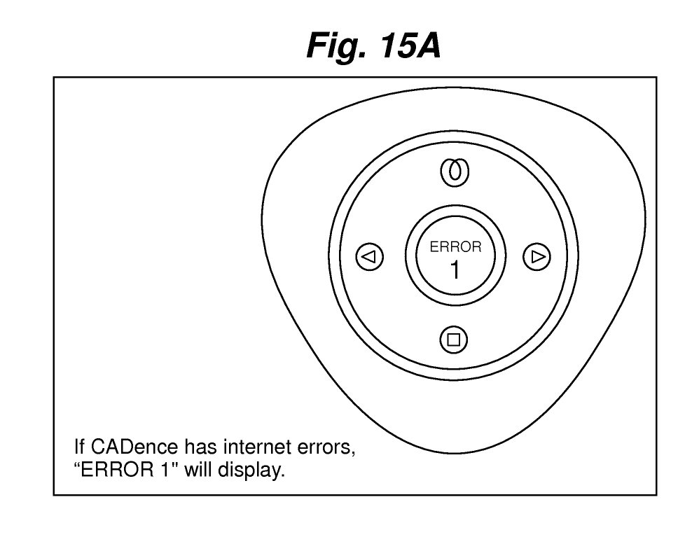

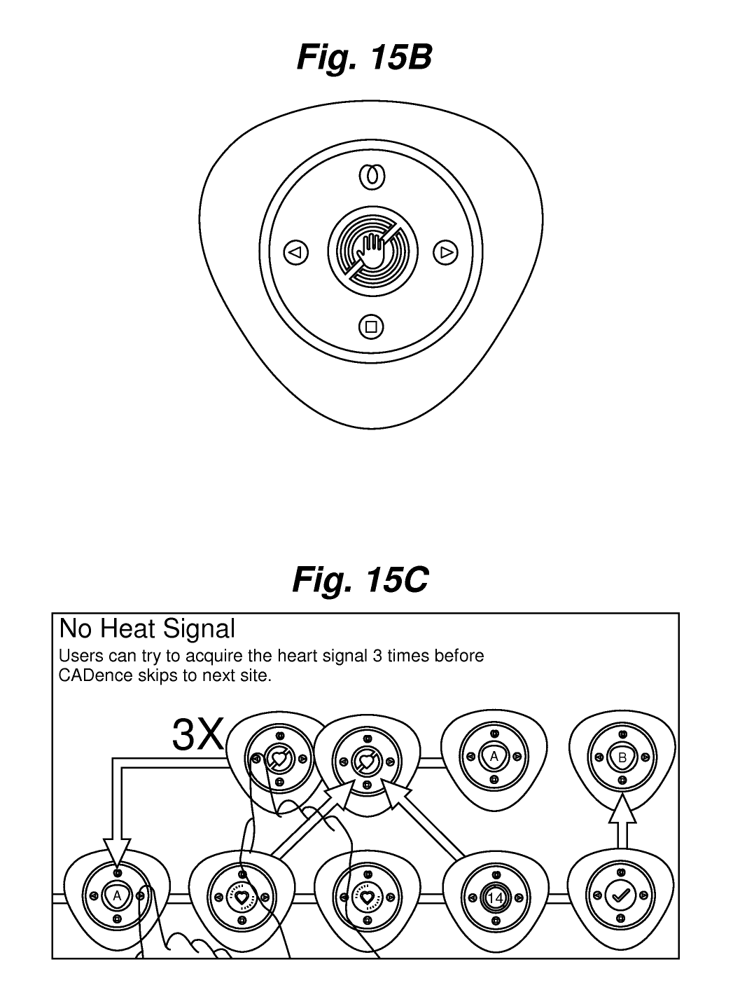

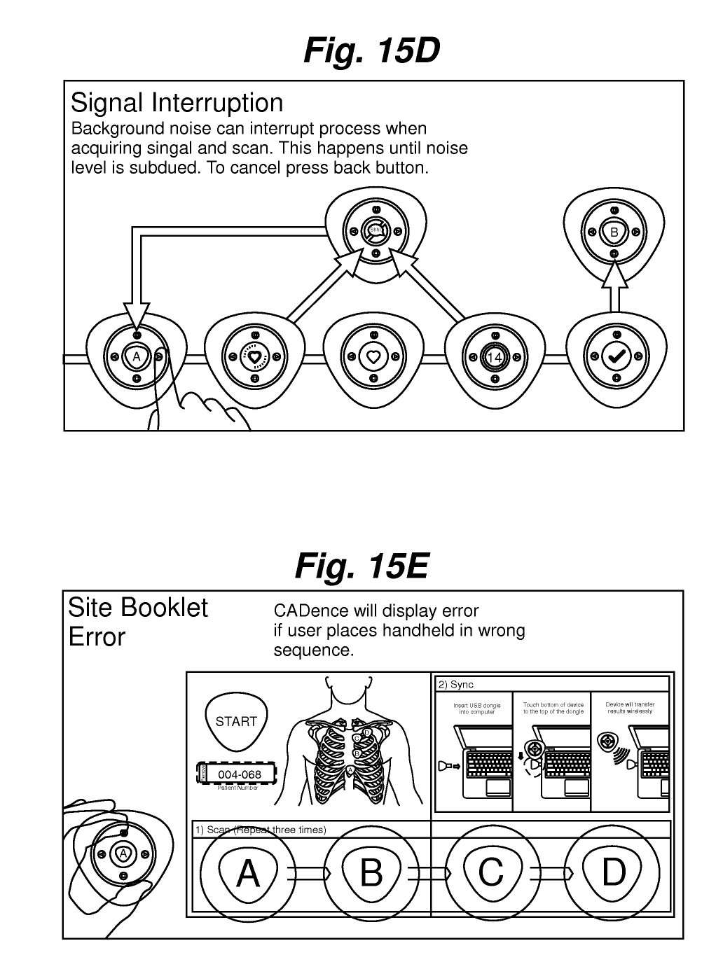

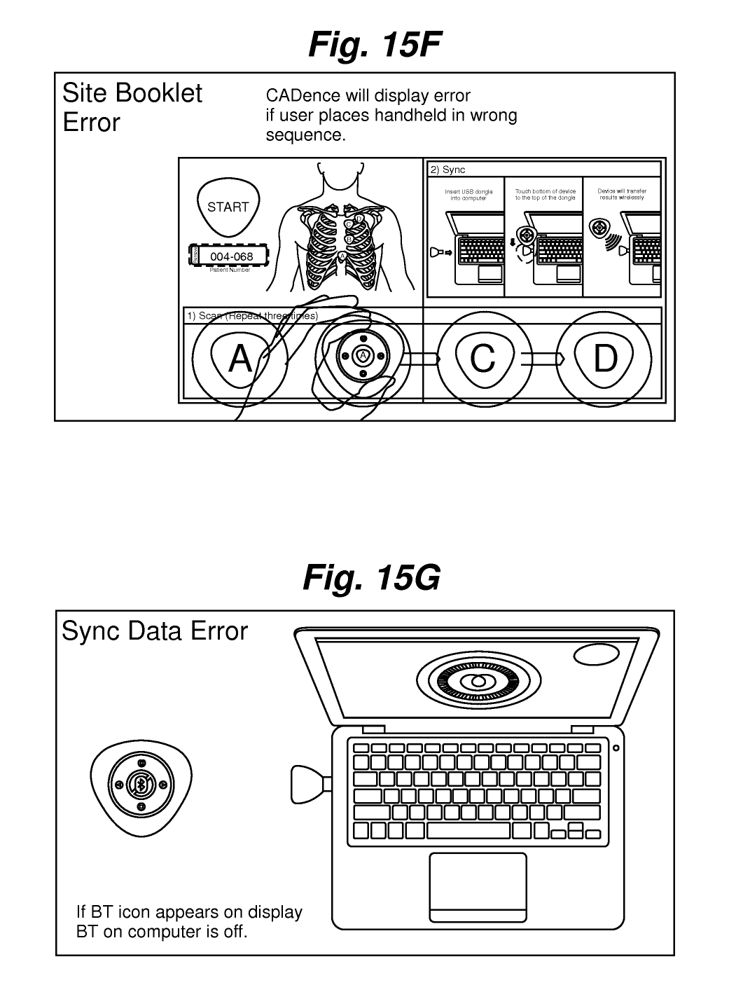

In embodiments, sequence guide 500 such as the one depicted in FIG. 8A can be coated in an anti-microbial coating for protection against disease transmission. In embodiments, anti-microbial materials can be embedded within the layers of sequence guide 500. Additional details relating to the operation of the CAD detection devices and corresponding sequence guides are described with respect to FIGS. 14 and 15.

In some settings, such as a clinic in which tests will be routinely performed, sequence guide 500 could be provided in a different format, such as a wall-hung poster or handheld, laminated card. In embodiments, the RFID or other signal associated with the steps shown in sequence guide 500 (“A” through “D”) do not vary between tests. In these embodiments, a single poster can be provided for use with the CAD detection device 400 over multiple tests. The Patient Number, however, can be different for each test subject. A variety of solutions are available to provide the Patient Number to the CAD detection device 400. These include “lottery style” tickets that each test subject receives, in which the RFID or other chip is housed within packaging that can only be open destructively, for example. Providing multiple Patient Number identifiers with a single sequence guide 500 can improve cost-efficiency for the test provider while maintaining anonymity of the health care data.

In an embodiment, referring specifically to FIG. 8B, sequence guide 500 is initially folded or otherwise secured so that two or more sides can be secured with a seal 502. In an embodiment, a broken seal indicates use of sequence guide 500. When seal 502 is broken, the set of CAD detection device 400 and/or sequence guide 500 can be discarded as previously used. In another embodiment, sequence guide 500 comprises seal 502 including a wireless chip, such as an RFID chip, NFC chip, or other suitable hardware, to signal initiation to CAD detection device 400. Activation of the chip or seal 502 can command CAD detection device 400 to begin initial processing prior to data collection.

In another embodiment, sequence guide 500 can be electromagnetically shielded such that data from wireless chips cannot be read from outside sequence guide 500. In an embodiment, the cover of sequence guide 500 is integrated with a shield. In an embodiment, the shield comprises conductive or magnetic materials, such as sheet metal, metal screen, and metal foam, or electromagnetically integrated paint or other coating.

In an embodiment of manufacturing sequence guide 500, RFID, NFC, or other wireless chips can be laid in a strip on the relevant scanning area identification pads. Such strip-based manufacturing saves time and money in producing sequence guide 500.

In an embodiment, sequence guide 500 can be integrated with a single removable patch. The patch can be removed from its coupling to sequence guide 500 and applied to a patient. In an embodiment, the patch comprises a sensor, circuitry, guide portion or other component at each relative location necessary to make a determination on the disease the system is intended to diagnose, such as coronary artery disease. For example, a single patch can be shaped to encompass all four locations (for example, those shown in FIG. 8A in the illustration of the patient chest) to determine coronary artery disease. A single “sample” event can then be conducted with all four locations once the patch is applied to the patient.

Sequence guide 500 can further comprise, in an embodiment, a display screen for displaying the results of the analysis or diagnosis of a particular disease. In other embodiments, a portion of sequence guide 500 comprises invisible ink or a heads-up display that can likewise display the results of the analysis or diagnosis of a particular disease.

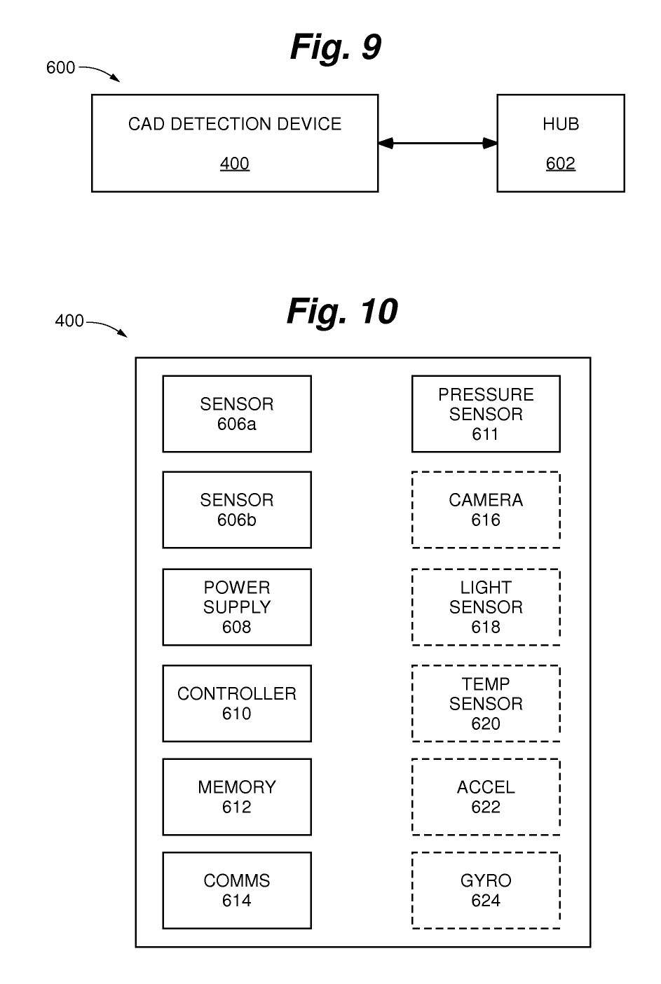

Referring to FIGS. 9 and 10, a system 600 for the detection of coronary artery disease is depicted. In an embodiment, system 600 generally comprises CAD detection device 400 and data portal device 602. Though not shown, in another embodiment, system 600 can further comprise a patient scan sequence guide 500. In other embodiments, system 600 can be operably coupled to a networked or local computer system similar to networked or local computer system 106 as discussed above with respect to FIGS. 1-4.

CAD detection device 400, as described above, comprises a handheld detection device that can be used in a non-invasive manner to determine whether an internal coronary artery blockage is present.

In an embodiment, referring to FIG. 10, CAD detection device 400 comprises at least one sensor 606, a power supply 608, a controller 610, a pressure sensor 611, a memory 612, and communications circuitry 614. CAD detection device 400 can comprise more or fewer components in various embodiments, examples of which are shown in dashed lines. In the embodiment shown in FIG. 10, these include camera 616, light sensor 618, temperature sensor 620, accelerometer(s) 622, and gyroscope(s) 624. These and other sensors can be used to determine the position, orientation, and/or acceleration of CAD detection device 400, or other details regarding ambient light or sound conditions, for example.

In the embodiment depicted in FIG. 10, CAD detection device 400 comprises two pressure sensors 606a and 606b. Sensors 606a and 606b can comprise pressure, acoustic, or other sensor modalities, with pressure sensors used in example embodiments discussed herein.

One pressure sensor 606a collects data related to the presence or absence of a turbulent pressure wave in a coronary artery. Blood flow is periodic in time and laminar in flow. A blockage can act as a nozzle within an artery, causing turbulence to occur. This turbulence creates a turbulent pressure wave that can be detected by pressure sensor 606a. Pressure sensor 606b, in one embodiment, is included for noise cancelling, such as active and/or passive noise cancelling to filter out ambient noise and other irrelevant information.

In other embodiments, one or both of sensors 606a and 606b can comprise acoustic or other sensors suitable for data collection, active and/or passive noise cancellation and/or other tasks related to the operation of CAD detection device 400. For example, in one embodiment of noise cancellation, background noise is sampled by at least one sensor 606a or 606b and subtracted from an overall signal in order to cancel noise and improve signal quality. In this and other embodiments, one or both of sensors 606a and 606b and/or related components and circuitry are mechanically potted, or encapsulated, to improve noise cancellation. In embodiments, one or both of sensors 606a and 606b are electrically shielded to reduce noise. Still other sensors in addition to sensors 606a and 606b also can be included in device 400, even though they are not specifically depicted in FIG. 10.

Automatic signal strength detection circuitry and signal processing techniques can also be implemented in embodiments to improve signal quality. For example, in one embodiment, a signal kernel or shape is determined from signal samples and compared with an overall signal or pattern to determine whether the sample fits. If not, new data can be collected, existing data can be otherwise processed, and/or detection device 400 can be repositioned, among other tasks.

Therefore, active noise cancellation can be implemented in embodiments of CAD detection device 400 as described above. Passive noise cancellation can be implemented in combination with active noise cancellation, or on its own, in other embodiments. Materials such as, but not limited to, rubber, foam, sponge, glass fiber, ceramic fiber, mineral fiber, vinyl, tape, or sound-absorbing coatings and pastes or combinations thereof can be disposed proximate one or both of sensors 606a and 606b or otherwise suitably arranged on or within device 400 in order to passively reduce noise.

In embodiments, one or both of sensors 606a and 606b or another sensor of device 400 can comprise pressure sensors or other sensors suitable for sensing the pressure applied by a user to CAD detection device 400 in preparation for or during patient scanning. In embodiments, one or both of sensors 606a and 606b can comprise pressure sensors or other sensors suitable for sensing the pressure applied by a user to detection device 400 in combination with the ability to collect data related to the presence or absence of a turbulent pressure wave in a coronary artery. In other embodiments, an additional sensor or plurality of sensors is configured for sensing the pressure applied by a user to detection device 400 in preparation for or during patient scanning.

In embodiments, the pressure applied by a user is measured and compared against a minimum value representative of a typical amount of minimum pressure to establish a signal, and a maximum value representative of a typical amount of maximum pressure so as to not max out the measured signal. If the measured pressure exceeds the maximum value, an error message can be displayed to the user via a user interface. Likewise, if the measured pressure is below the minimum value, an error message can be displayed to the user via the user interface. Other threshold minimum and maximum values can also be used, depending on the patient, user, or other appropriate factors.

Power supply 608 can comprise a battery in embodiments, such as a rechargeable battery or a replaceable battery. Rechargeable power supply 608 can be inductance-style, two-pin, charge by computer, and/or charge by AC wall outlet, or some other suitable charging configuration. In embodiments, power supply 608 can be powered or recharged through inductive charging. Power supply 608 can also be configured to allow for multiple different charging schemes. For example, CAD detection device 400 can interface with a charging station by physical coupling or cable, and/or CAD detection device 400 can couple by USB or other cable to a computer, docking station, wall outlet or other source of power. In embodiments, one power supply 608 is electrically shielded to reduce noise.

Controller 610 controls the operation of CAD detection device 400. During scanning, controller 610 can control a graphical user interface (GUI), a timer and the general operation of CAD detection device 400. In embodiments, the GUI is electrically shielded to reduce noise. Controller 610, via the GUI and/or an audible indicator, can also prompt a user to carry out various tasks, such as to apply CAD detection device 400 to one or more of patient scan sequence guide 500 areas or patient areas, to move on to a next guide area, tag or patient area, to rescan a particular guide area, tag or patient area, to reposition CAD detection device 400 if data of sufficient quality is not detected, to recharge CAD detection device 400 and other functions. CAD detection device 400 further comprises a timer, such as part of controller 610, for each scan to ensure that sufficient data is collected at each scan site. In one embodiment, this timer can automatically start as soon as data of a sufficient quality is detected by sensor 606a, though other procedures can be used in other embodiments.

Controller 610 can be subservient to and/or operate in conjunction with an external controller coupled by wire or wirelessly in embodiments. In embodiments, controller 610 can carry out processing of collected data, for example to determine a presence of coronary artery disease based on data sampled by at least one of sensors 606a and 606b at the patient data acquisition locations associated with identification areas guide 500, though in other embodiments data processing is carried out external to CAD detection device 400, as described above. Controller 610 operates in cooperation with memory 612, which stores collected data until it is transferred to an external device or manually or automatically deleted and can be of any suitable type. Controller 610 also can correlate data sampled by sensors 606a and/or 606b with a data acquisition location using the identification element, such as sequence guide 500.

Communication circuitry 614 is configured to transfer data to and from CAD detection device 400. For example, after scan data is collected for a particular patient and stored in memory 612, the raw scan data can be packaged and transferred wired or wirelessly to, for example, data portal device 104 or networked or local computer system 106 for processing and determination of whether CAD may be present.

In another embodiment, CAD detection device 400 optionally further comprises a camera 616, photo or optical sensor, or other similar device. In embodiments, camera 616 can be configured to read or detect barcodes, such as one or more QR codes on sequence guide 500. In an embodiment, instead of visually moving from “A-B-C-D” as part of the sequence scanning, a QR code can be scanned by camera 616 and the subsequent sensing of the appropriate portion of the patient then conducted in any order. In embodiments, camera 616 is further configured for detecting or aligning to a portion of the patient's body. In embodiments, camera 616, along with appropriate software executable by controller 610, is configured to detect markers, flags, or any other suitable landmark of the patient to aid in aligning CAD detection device 400 to the patient. In another embodiment, camera 616 is configured to detect position of motion relative to the patient.

In another embodiment, CAD detection device 400 optionally further comprises a light sensor 618. In embodiments, light sensor 618 can be utilized to detect the proximity of CAD detection device 400 to the body of the patient. For example, a greater amount of light will be detectable by light sensor 618 when CAD detection device 400 is further from the patient. Likewise, less light will be detectable by light sensor 618 when CAD detection device 400 is proximate the patient. Such light sensing can be utilized to properly place and position CAD detection device 400.

In another embodiment, CAD detection device 400 optionally further comprises a temperature sensor 620. In embodiments, temperature sensor 620 can be utilized to detect the proximity of CAD detection device 400 to the body of the patient. For example, a lower temperature will be detectable by temperature sensor 620 when CAD detection device 400 is further from the patient. Likewise, a higher temperature will be detectable by temperature sensor 620 when CAD detection device 400 is proximate the patient. Such temperature sensing can be utilized to properly place and position CAD detection device 400. In embodiments, temperature sensor 602 can be used to measure the temperature of a patient.

In another embodiment, CAD detection device 400 optionally further comprises one or more accelerometers 622. In embodiments, accelerometer 622 is configured to detect proper acceleration of CAD detection device 400. For example, if accelerometer 622 detects an acceleration when CAD detection device 400 is in a data sense mode, it can be determined that CAD detection device 400 has moved or shifted during sensing and the data measured during that point may have errors or otherwise be incorrect. Such acceleration sensing can be utilized to properly place and position CAD detection device 400. In embodiments, an indication of a shift can be provided to the user via the GUI of CAD detection device 400. In still further embodiments, accelerometers 622 can provide drop detection. If CAD detection device 400 is dropped and accelerometers 622 detect sharp acceleration, CAD detection device 400 can be deactivated so that results from a damaged device 400 are not used in diagnosis. In embodiments, a detected drop can be transmitted to a licensor or owner of the equipment.

In an embodiment, CAD detection device 400 optionally further comprises one or more gyroscopes 624. In embodiments, gyroscope 624 is configured to detect an orientation or change in orientation of CAD detection device 400. For example, if gyroscope 624 detects a change in orientation of CAD detection device 400 when CAD detection device 400 is in a data sense mode, it can be determined that CAD detection device 400 has tilted during sensing and the data measured during that point may have errors or otherwise be incorrect. Such orientation sensing can be utilized to properly place and position CAD detection device 400. In embodiments, an indication of a tilt can be provided to the user via the GUI of CAD detection device 400.

In an embodiment of CAD detection device 400 comprising gyroscope 624, noise determination can be conducted by determination of orientation or movement sensed by gyroscope 624. In embodiments, based on the amount of movement sensed by gyroscope 624, additional determination can be made of the source of the noise, such as lung noise, digestive noise, or movement of the device. In such embodiments, CAD detection device 400 comprises a single sensor 606a, and does not include a secondary sensor for noise reduction or noise cancellation.

In an embodiment, CAD detection device 400 optionally further comprises an SD card port configured to receive an SD card or other portable memory. In such embodiments, CAD detection device 400 is effectively infinitely expandable such that an unlimited number of data sets can be stored. In other embodiments, an internal memory such as MANIC can be incorporated, which is not removable from CAD detection device 400.

In embodiments, CAD detection device 400 can be configured for gain control. In an embodiment, gain control can be automated. For example, an impedance measurement can detect abdominal loading or feedback and the gain adjusted appropriately. Gain can therefore be modified based on an impedance feedback. In embodiments, gain tuning or control can be based at least in part on data downloaded from an Electronic Health Record (EHR). In embodiments, CAD detection device 400 can be pre-calibrated before use.

Embodiments of CAD detection device 400 can be utilized with existing systems to provide supplemental functionality to those systems. For example, CAD detection device 400 can be operably coupled to an electrocardiography (ECG or EKG) device. Data sensed or detected by CAD detection device 400 can supplement electrocardiogram data. In embodiments, the supplemented or combined data can be incorporated into a diagnosis or used in further data analysis. In embodiments, CAD detection device 400 can be operably coupled to other existing systems.

Referring again to FIG. 9, data portal device/hub 602 comprises a device configured to operably couple CAD detection device 400 to a remote server without the use of an intermediary computer. In an embodiment, hub 602 is configured for manual or automatic data transfer and carried out via WIFI, BLUETOOTH, low-power BLUETOOTH, RFID, NFC, mini-USB, Ethernet or other cable, cellular signal or some other suitable wired or wireless data push methodology. Hub 602 therefore allows for the pairing of CAD detection device 400 with networked or local computer system 106, without special protocol or hardware implementation.

In another embodiment, hub 602 further comprises a microphone or other suitable voice sensor such that system 600 can be verbally or audibly commanded with hub 602. For example, voice commands can be given to hub 602 to command CAD detection device 400, such as to turn on, turn off, activate, sense data, or otherwise command CAD detection device 400. In an embodiment, identifying patient data can be spoken into hub 602. In embodiments, the data sensed from CAD detection device 200 can be combined into a single package with the patient data spoken into hub 602.

In an embodiment, hub 602 can further comprise a user-mountable attachment, such as a bracelet or necklace. In such embodiments, hub 602 can be worn by the patient or caregiver. Hub 602 can be worn such that hub 602 is placed on the user to facilitate speaking or otherwise commanding system 600 or components of system 600 via voice or other audible commands.

In embodiments, a kit can comprise a CAD detection device 400 and a hub 602. In embodiments, a kit further comprises sequence guide 500. In other embodiments, a kit can further comprise instructions for use of CAD detection device 400, hub 602, and/or sequence guide 500. In embodiments, sequence guide 500 contains instructions for use of CAD detection device 400 and hub 602.

In embodiments, drivers, software, and other executables, if necessary, can be installed on hub 602. In one embodiment, data transfer from CAD detection device 400 to hub 604 is initiated using RFID technology embedded in a USB Bluetooth hub 602. The hub drivers can be pre-installed, in embodiments.

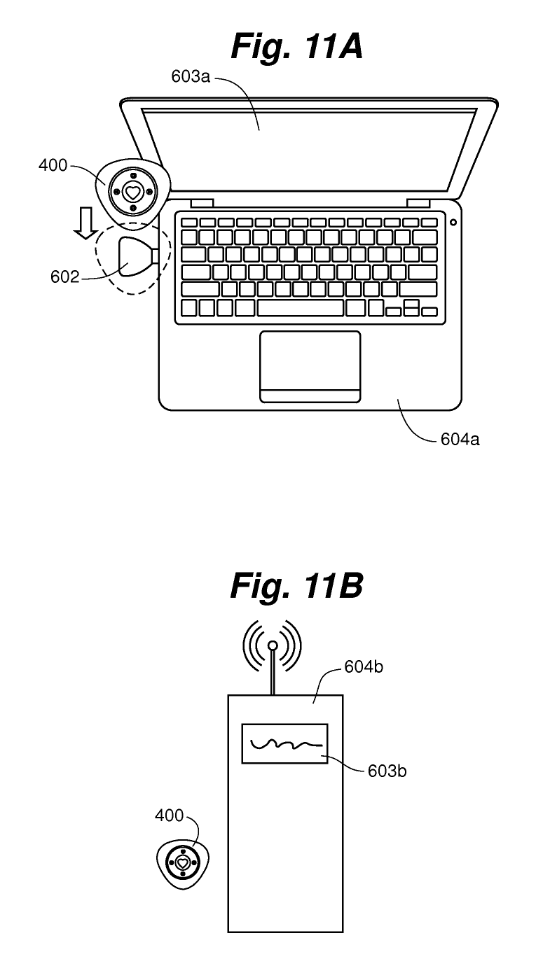

Referring to FIGS. 11A-11B, data is transferred using secure wireless communications and software to a networked or local computer systems (e.g., networked or local computer system 106). In the embodiment shown in FIG. 11A, data is transferred to dongle 602 from CAD detection device 400 using secure wireless communications and software, and then to host computer 604a, where the data can be processed or subsequently forwarded to networked or local computer system 106.

Dongle 602 is one possible physical embodiment of a hub or data portal device, as previously described, although in alternative embodiments various other structures could be used either with or without a host computer 604a to transfer information to or from a remote server. For example, in the embodiment shown in FIG. 10B, dongle 602 and host computer 604a are absent, and an always-on, wirelessly connected hub 604b is configured to receive data from CAD detection device 400 and transmit that information to a remote server such as networked or local computer system 106. In some embodiments, hub 604b can be plugged into a wall outlet or other power source, and in other embodiments hub 604b can be powered by a battery or other power source. Hub 604b can transmit via any of a variety of wireless communication means, including but not limited to Wi-Fi, cellular transmission, or Bluetooth. In some embodiments, hub 604b can transmit via more than one transmission medium, or can receive via one transmission medium (e.g., Wi-Fi) and transmit via another (e.g., cellular).

A test result, such as an indication of the presence or absence of coronary artery disease and/or a recommendation for further review or analysis, can be transferred back to CAD detection device 400 from networked or local computer system 106 or other remote server via communications circuitry and displayed on a display (e.g., display 603a of FIG. 11A or display 603b of FIG. 11B). In other embodiments, scan results can be retrieved remotely by a medical professional, securely emailed to a medical professional, transferred to an electronic health record or otherwise suitably communicated to a medical professional and/or patient. In an embodiment, the data transfer process can be initiated by the CAD detection device 400 via a scan of an RFID tag embedded in dongle 602. In other embodiments, results can instead be communicated to or retrieved by a desktop or laptop computer, a networked device, a portable medical device, a PDA, or some other suitable electronic device accessible by a medical professional or other user. The result itself can be presented in a variety of ways, such as by “+” or “−” indication on CAD detection device 400 or in a written report, such as in PDF or other digital or hardcopy form, with more detailed information, analysis and next-steps recommendations, among others.

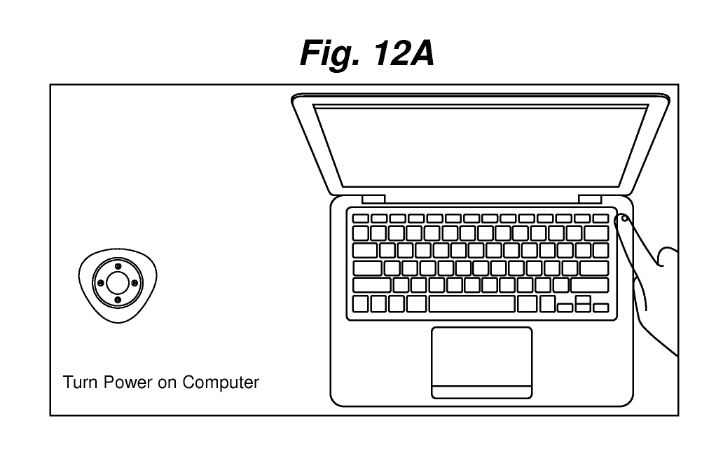

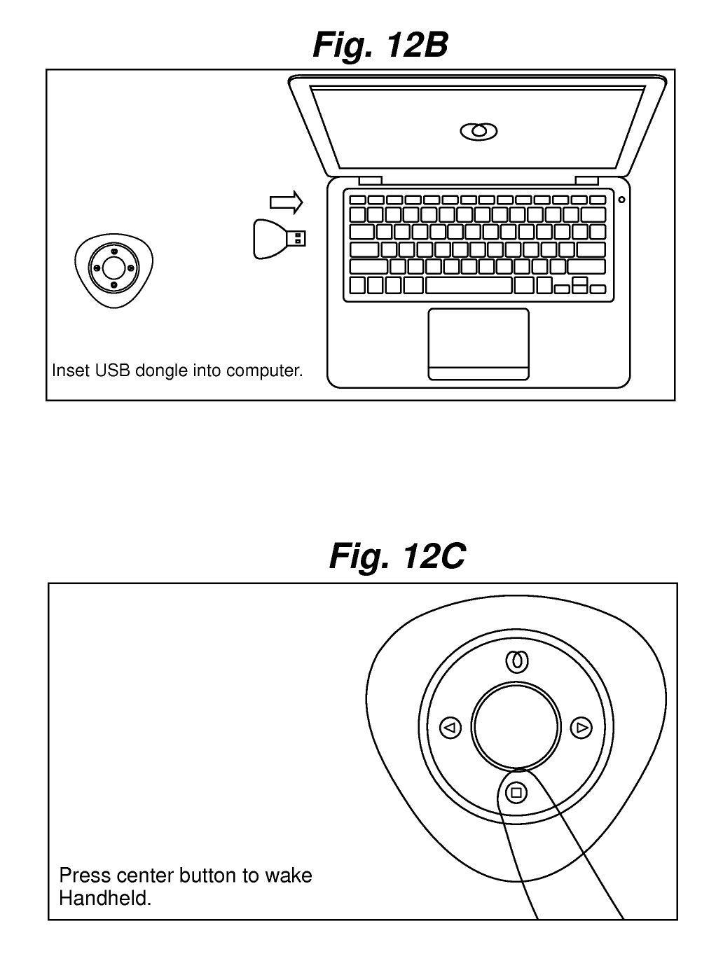

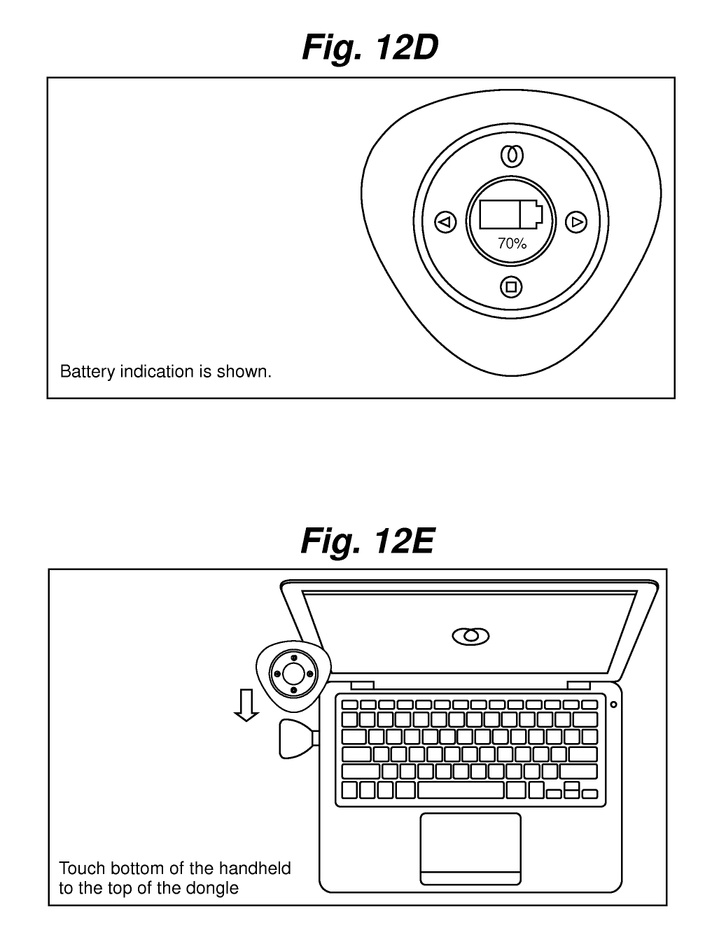

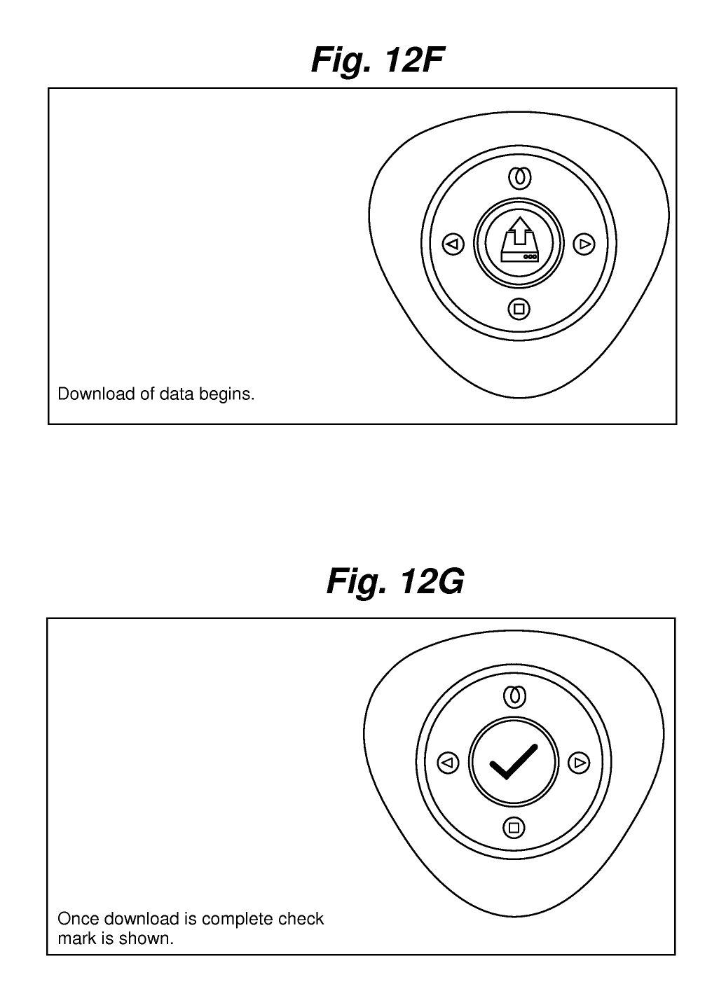

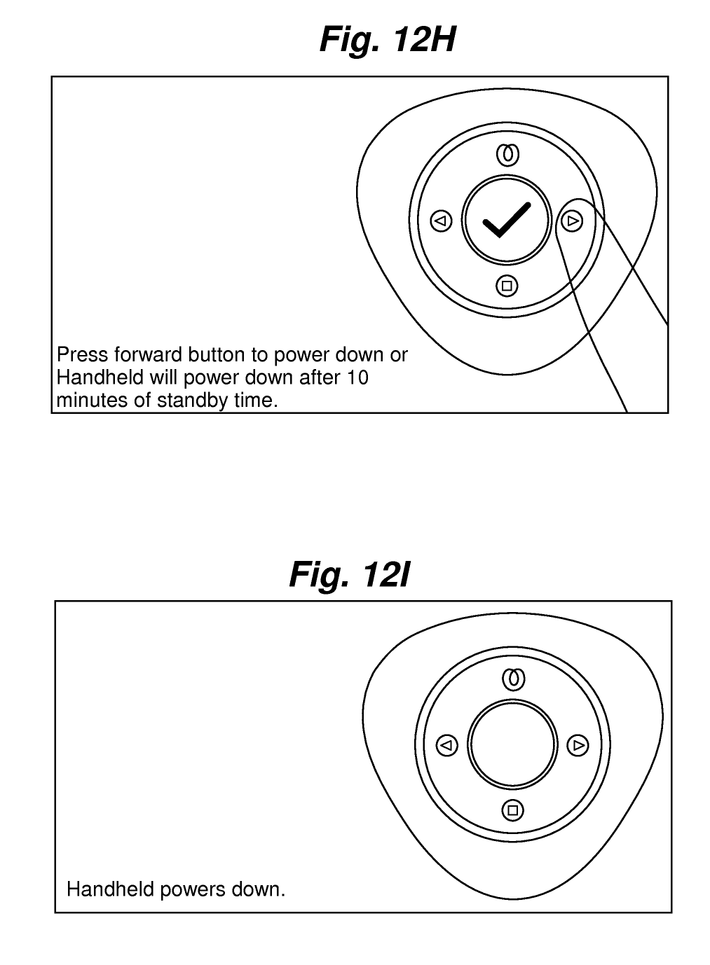

Referring to FIGS. 12A-12I, an example of one usage cycle of CAD detection device 400 is depicted. In the example shown in FIGS. 12A-12I, a dongle 602 and host computer 604 are shown, although in alternative embodiments a hub (e.g., 604a) could be used instead of those two components.

Referring first to FIG. 12A, a computer such as host computer 604 can be turned on. Referring to FIG. 12B, a dongle such as dongle 602 can be inserted into host computer 604. Referring to FIG. 12C, a handheld device such as CAD detection device 400 can be activated by pressing the center button. Activation of CAD detection device 400 can cause an auto-synchronization routine to run, connecting CAD detection device 400 with dongle 602 and host computer 604. In an embodiment, referring to FIG. 12D, a battery status indication is shown when the handheld device is activated or woken from sleep. In alternative embodiments, a number of tests remaining before recharging is required can be shown when the handheld device is activated or woken from sleep. Referring to FIG. 12E, CAD detection device 400 can be contacted with dongle 602. In alternative embodiments where a hub or data portal device is used rather than a dongle, CAD detection device 400 can automatically sync with the hub or data portal device upon waking from sleep mode or when a scan is complete. As shown in FIG. 12F, data download from CAD detection device 400 to dongle 602 begins. A status is shown on CAD detection device 400. Once the download from CAD detection device 400 to dongle 602 is complete, a check mark or other confirmation can be presented on CAD detection device 400. In other embodiments, a confirmation is presented on dongle 602 (not shown). Optionally, referring to FIG. 12H, a forward button can be pressed to power down CAD detection device 400. In another embodiment, CAD detection device 400 powers down after 10 minutes of standby time. In other embodiments, CAD detection device 400 can be configured to power down after less than 10 minutes or more than 10 minutes. For example, CAD detection device 400 is shown powered down in FIG. 12I.

A data portal device or hub can be used to carry out those activities shown in FIGS. 12A-12I, rather than a combination of a dongle and host computer. In embodiments, a data hub or data portal device can be wirelessly accessible, and can also be smaller and can be positioned out of the way of patient testing. Accordingly, when the patient testing space is small, crowded, contains potential contaminants, or is otherwise an unsuitable work environment such as a barn or animal pen, use of a data hub or data portal device can free up valuable exam space that would otherwise be occupied by the host computer.

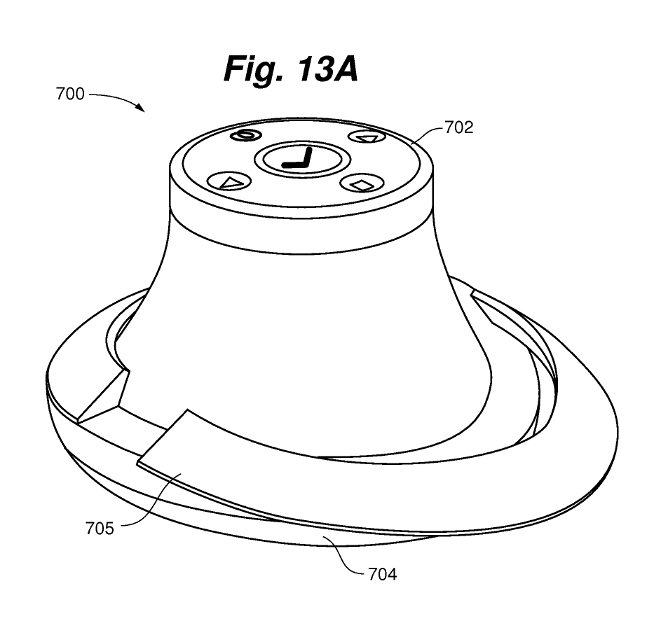

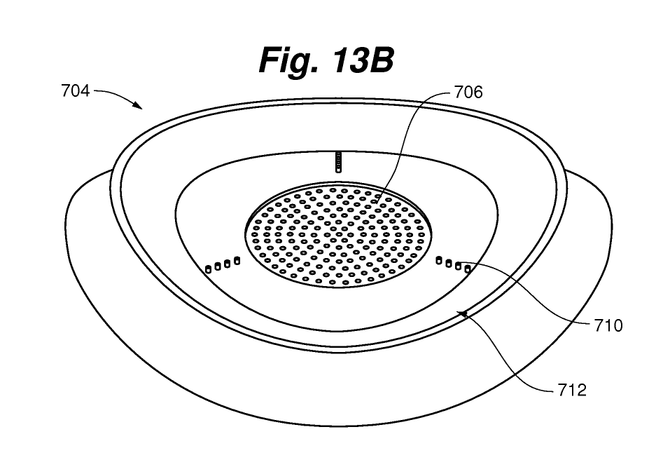

Referring to FIG. 13A, a system 700 for the detection of coronary artery disease is depicted. System 700 generally comprises CAD detection device 702 and docking station 704. CAD detection device 702 is substantially similar to CAD detection device 400.

Docking station 704 comprises hardware configured to receive one or more CAD detection devices 702. In an embodiment, docking station 704 is configured to receive CAD detection device 702 by a physical plugging, wiring or other coupling. Tab 705 closes around at least a portion of CAD detection device 702 to promote good contact between CAD detection device 702 and docking station 704. In addition, tab 705 can reduce or eliminate light, noise, and/or vibration that could otherwise affect communication between CAD detection device 702 and docking station 704.

In embodiments, CAD detection device 702 can communicate with docking station 704 to perform a self-test, calibration, or other diagnostic functions. These diagnostic features of docking station 704 can be used to assess the functionality of any of the sensors (e.g., sensors 606-624 previously described with respect to FIG. 9). In embodiments including an acoustic sensor, tab 705 can beneficially reduce ambient noise that could otherwise result in diagnostic errors.

In embodiments, CAD detection device 702 further comprises a hardware port for data transfer. Likewise, docking station 704 comprises a corresponding port for data transfer. In other embodiments, docking station 704 is configured to receive CAD detection device 702 by a wireless connection, such as a near-field or RFID communication. In embodiments, docking station 704 is further configured to charge or recharge a power supply of CAD detection device 702, such as power supply 608 as discussed with respect to CAD detection device 400. The relative orientation of device 702 and docking station 704 to accomplish docking can be different than that depicted in FIG. 13.

In operation of docking station 704, once CAD detection device 702 has completed data collection as described above, CAD detection device 702 can be plugged into, docked, or otherwise coupled to docking station 704. Docking station 704 can then receive the collected data. Once received, in embodiments, docking station 704 can be configured to analyze the data. In other embodiments, as described above with respect to FIGS. 1-4, docking station 704 can forward on the data to, for example, data portal device 104 and/or networked or local computer system 106. Docking station 704 is further configured to receive the results or a diagnosis based on the analyzed data.

Docking station 704 is shown in more detail in FIG. 13B. In the embodiment shown in FIG. 13B, tab 705 and CAD detection device 702 are not shown in order to depict underlying features of docking station 704. In the embodiment of docking station 704 shown in FIG. 13B, speaker 706 and pins 710 are positioned within a dish portion 712 of docking station 704.

Speaker 706 can emit acoustic vibrations for testing of CAD detection device 702. When docked, as shown in FIG. 12A, CAD detection device 702 can receive acoustic signals emitted by speaker 706. By comparing the received acoustic signals at CAD detection device 702 with those emitted by docking station 704, CAD detection device can be calibrated. Alternatively, where CAD detection device 702 is unable to detect acoustic signals accurately, CAD detection device 702 can be diagnosed and/or an indication that a repair or replacement is needed can be generated.

In embodiments, pins 710 can be used to route signal from CAD detection device 702 to docking station 704. These signals can include, for example, the detected signals during a self-test or calibration procedure, or the signals acquired during a test of a patient. Additionally or alternatively, pins 710 can be used to charge a battery in CAD detection device 702.

In alternative embodiments, the relative positions of speaker 706, electrical contact pads 708, and/or pins 710 can be changed within dish portion 712. Furthermore, dish portion 712 can have a different size or shape than that which is shown in FIG. 13B. In embodiments, dish portion 712 can be shaped to correspond with the shape of CAD detection device 702.

In some embodiments, self-testing of CAD detection device 702 can be required by the software on CAD detection device 702 before an additional test can be run. Depending on the type and frequency of testing, CAD detection device can be diagnosed and calibrated by docking station 704 before every test, or before every other test, or before every third test, etc. In some embodiments, a diagnostic test can be required after a certain amount of time has elapsed, no matter how many intervening patient tests have been performed. In embodiments, a remote user could send a request via internet to hub, then via Bluetooth to the device 702 to perform a sensor test. The user could communicate to the CAD detection device 702 via wireless Internet, for example, in embodiments where CAD detection device 702 includes a chip that allows it to connect to the Internet. The user could also upgrade the firmware remotely via Over The Air (OTA) technology.

Referring to FIGS. 14A-14N, an embodiment of a CAD detection device 800 is depicted in operation with a scanning guide 802. In embodiments, CAD detection device 800 is substantially similar to CAD detection device 400. In embodiments, scanning guide 802 is substantially similar to scanning guide 500.

As shown, CAD detection device 800 can further comprise a display or screen. In embodiments, the display can comprise OLED monochrome, full color or a heads-up projected display. In embodiments, the number of LED lights activated can be used to indicate a status of CAD detection device 800. In embodiments, CAD detection device 800 can further comprise a graphics card configured to operate or present on the display or screen. In embodiments, the display or screen is a touchscreen. In embodiments, the display or screen comprises a membrane switch. In another embodiment, the display or screen comprises a capacitive screen.

Referring to FIG. 14A, in an embodiment, upon power up, a home screen is displayed on CAD detection device 800. In an embodiment, the home screen is displayed for 3 seconds. In other embodiments, the home screen is displayed for greater than or less than 3 seconds. In an embodiment, an audible “ready” sound can further be provided from CAD detection device 800.

Referring to FIG. 14B, in an embodiment, after the home screen is displayed, a “Start” screen is displayed indicating that the patient scan can begin. In an embodiment, an audible “Start” sound can further be provided from CAD detection device 800.

Referring to FIG. 14C, in an embodiment, CAD detection device 800 can be positioned near scanning guide 802 in preparation for use.

Referring to FIG. 14D, in an embodiment, CAD detection device 800 can be positioned on the “Start” RFID tag (See FIG. 13C) to digitally save the patient information.

Referring to FIG. 14E, in an embodiment, CAD detection device 800 screen can display a green color or indicator to signal that the patient information was successfully saved on the memory of detection device 800. In an embodiment, the screen color is changed for a duration of 1 second. In other embodiments, the screen color is changed for greater than or less than 1 second.

Referring to FIG. 14F, in an embodiment, CAD detection device 800 screen can change to indicate that the user touch Site A to begin testing.

Referring to FIG. 14G, in an embodiment, the user can place CAD detection device 800 at Site A. Such placement links CAD detection device 800 with the specified scan sequence (i.e. beginning with Site A).

Referring to FIG. 14H, in an embodiment, CAD detection device 800 screen can change to an “A” and an arrow indicating the user to press the arrow to start.

Referring to FIG. 14I, in an embodiment, the user can position CAD detection device 800 on Site A of the user.

Referring to FIG. 14J, in an embodiment, the user can steady CAD detection device 800 on Site A of the user and press the aforementioned forward arrow to start the scan. In embodiments, this can be accomplished with one hand, such that buttons on CAD detection device 800 do not need to be pressed firmly. In other embodiments, two hands are utilized. In still further embodiments, the orientation of CAD detection device 800 can be determined internally based on the position of the device 800. Therefore, in such embodiments, it may not be necessary in these embodiments to orient the device in any particular way. Furthermore, in some embodiments, rather than pressing a button to begin testing, the audio data corresponding to a heartbeat can activate recording.

Referring to FIG. 14K, once the forward arrow is pressed and the scan is started for Site A, CAD detection device 800 is configured to search for a signal heartbeat. In an embodiment, the display is blue if a heartbeat signal is found. In an embodiment, the display is red if a heartbeat signal is not found. Such feedback features provide an immediate indication of the success or failure of the detection. In embodiments, a secondary sensor configured to detect ambient noise as described above with respect to CAD detection device 400 provides immediate instruction to stop data collection. In embodiments, the display can be updated once per second. In other embodiments, the display can be updated more or less frequently. In embodiments, if there is no heartbeat found for 7 seconds, CAD detection device 800 times out. In other embodiments, CAD detection device 800 can time out after more or less than 7 seconds.

In embodiments, multiple attempts are provided at each site to successfully record a heartbeat signal and/or the associated data. If CAD detection device 800 is unsuccessful after a number of tries, the system moves on to the next site. Such site progression is discussed further with respect to FIG. 15C. In other embodiments, after three unsuccessful tries, the entire scan sequence is halted. In other embodiments the user may contine to try to collect data until the site has been collected.

Referring to FIG. 14L, CAD detection device 800 screen can display an indication that a signal is found. In an embodiment, the screen can display a green heart shape. In embodiments, the indication is displayed for 1 second. In other embodiments, the indication can be displayed shorter or longer than 1 second. In embodiments, an audible “success” sound can further be provided from CAD detection device 800.

Referring to FIG. 14M, once a heartbeat is found, the scan can begin and a countdown can be displayed on the screen of the CAD detection device 800. In an embodiment, a scan takes a duration of about 20 seconds. In embodiments, as shown in FIG. 14M, if a heartbeat signal continues to be detected, the countdown display can be in blue. In embodiments, if the heartbeat signal is not found, the countdown display can be in red. In embodiments, the display can be updated once per second. In other embodiments, the display can be updated more or less frequently. In other embodiments (not shown), a timer-based progress bar is not shown. Instead, a simple numbered countdown is displayed.

Referring to FIG. 14N, once the scan is successful, a success indication can be provided. In embodiments, the indication is displayed for 1 second. In other embodiments, the indication can be displayed shorter or longer than 1 second. In embodiments, an audible “success” sound can further be provided from CAD detection device 800.

In embodiments, the aforementioned sequence of FIGS. 14F-14N can be repeated for sites B, C, and D.

In embodiments, CAD detection device 800 can display a number of error messages. For example, referring to FIG. 15A, if CAD detection device 800 contains internal errors, such as with any of the components described above with respect to CAD detection device 400, an “ERROR 1” will display. In other embodiments, other errors can display to indicate the subcomponent or subcomponents that have failed.

Referring to FIG. 15B, if during a scan, the user is applying too much force to CAD detection device 800, a pressure error can be indicated.

Referring to FIG. 15C, in an embodiment as described above, a user can attempt to acquire a heart signal three times before CAD detection device 800 proceeds to the next site. Error handling and error display is provided for the signal acquisition, as illustrated.

Referring to FIG. 15D, in an embodiment, if background noise interrupts the acquisition signal and scan, error handling and error display is provided. For example, until the noise level is removed or reduced, the scan is interrupted. In an embodiment, the user can press the “back” button on CAD detection device 800 to cancel the site scan.

Referring to FIGS. 15E-15F, in an embodiment, if the user places CAD detection device 800 in the wrong site on sequence guide 802, an error can be displayed on CAD detection device 800.

Referring to FIG. 15G, in an embodiment of a system incorporating a dongle, a data sync error can be provided if data is unable to sync. For example, a Bluetooth icon can appear on the display if the host computer is turned off.

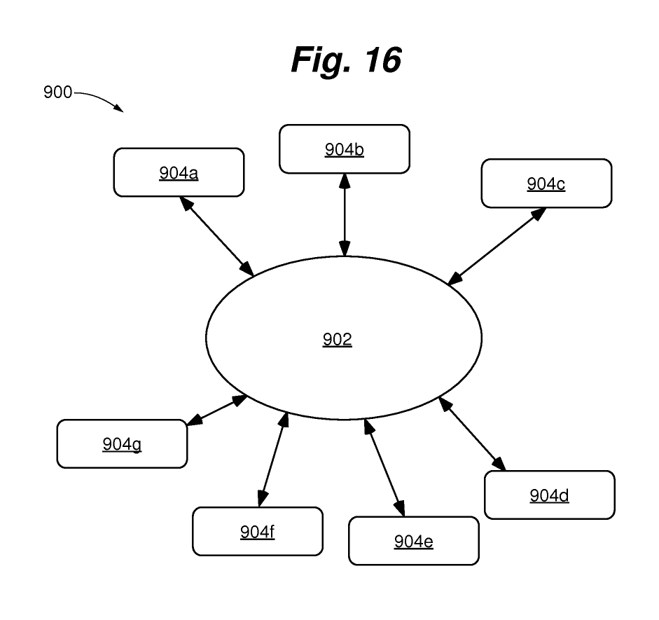

FIG. 16 depicts a neural network 900. Neural network 900 includes learning process 902 configured to receive and send data with each of a plurality of remote detection devices 904a-904g. As detection devices 904a-904g acquire data relating to tests of individual patients, that data can be transmitted to learning process 902. Learning process 902 can compare the data from each device to establish more accurate baselines and, if subsequent testing reveals that any of the tests were of a patient having a particular disease or disorder, learning process 902 can improve the algorithm or criteria used to analyze results from each of the detection devices 904a-904g. In embodiments, there can be more or fewer detection devices 904. Learning process 902 can also include a database for storing historical results, so that over time the detection algorithm(s) can be improved.

Any of the plurality of remote detection devices 904a-904g can compare a sensed data set to the stored data sets at the learning process 902 to determine a likelihood that a test subject has a coronary artery disease. The comparison can occur either at the detection devices 904a-904g, or the comparison can be made at a cloud-based server, as described with respect to FIGS. 1-4, in various embodiments.

Various embodiments of systems, devices, and methods have been described herein. These embodiments are given only by way of example and are not intended to limit the scope of the invention. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A detection system comprising:
a detection device having a plurality of sensors and configured to:
detect data corresponding to at least one anatomical or physiological characteristic, and
transmit the data;
a data portal device configured to receive the data from the detection device, the data portal device further configured to transmit the data; and
a networked or local computer system configured to receive the data from the data portal device, generate a diagnosis related to the at least one anatomical or physiological characteristic based on the data, and provide the diagnosis as an output.

2. The system of claim 1, wherein the at least one anatomical or physiological characteristic is one of the group consisting of:
cardiac anatomy, cardiac physiology, pulmonary anatomy, pulmonary physiology, valvular anatomy, valvular physiology, renal artery anatomy, renal artery physiology, peripheral artery anatomy, peripheral artery physiology, carotid artery anatomy, and carotid artery physiology.

3. The system of claim 1, further comprising a docking station configured to receive the detection device.

4. The system of claim 3, wherein the docking station is configured to wirelessly calibrate the detection device.

5. The system of claim 1, wherein the detection device comprises a handheld device.

6. The system of claim 1, wherein the detection device comprises a catheter.

7. The system of claim 1, wherein the detection device comprises an implantable device.

8. The system of claim 1, wherein at least one of the plurality of sensors is configured to sense the data, and wherein the detection device comprises:
a power supply configured to provide power to the sensor;
a controller configured to receive power from the power supply and to operate the sensor; and
communications circuitry configured to receive the data corresponding to the at least one anatomical or physiological characteristic and transmit the data to the data portal device.

9. The system of claim 1, wherein the detection device and the data portal device are configured to mechanically engage.

10. The system of claim 9, wherein the detection device is configured to transmit the data at substantially the same time that the data is detected.

11. The system of claim 8, wherein the communications circuitry comprises a wireless chip.

12. The system of claim 11, wherein the data portal device is a hub configured to receive and send wireless signals.

13. The system of claim 1, wherein the networked or local computer system comprises a learning process module.

14. The system of claim 1, wherein the networked or local computer system comprises a plurality of servers in different networked or local computer systems.

15. The system of claim 1 further comprising a database, wherein the data corresponding to an anatomical or physiological characteristic and the diagnosis are stored on the database.

16. The system of claim 15, wherein the database is a part of a learning network.

17. The system of claim 15, wherein the database is configured such that a user can access the data and the diagnosis.

18. A method for generating a diagnosis, the method comprising:
providing a detection device having a plurality of sensors and configured to detect data corresponding to at least one anatomical or physiological characteristic and transmit the data; and
providing a data portal device configured to receive the data from the detection device, the data portal device further configured to transmit the data to a networked or local computer system configured to generate a diagnosis related to the at least one anatomical or physiological characteristic based on the data and provide the diagnosis as an output;
transmitting the data from the data portal device to a remote server; and
calculating, at the server, a probability of an abnormality of the anatomical or physiological characteristic to generate the diagnosis.

19. The method of claim 18, wherein the detection device is configured to broadcast the data wirelessly.

20. The method of claim 18, wherein generating the diagnosis comprises calculating, at the networked or local computer system, a probability of an abnormality of the anatomical or physiological characteristic.

21. The method of claim 18, wherein the data portal device comprises a dedicated pass-through device.

22. The method of claim 18, wherein the transmitting from the data portal device and the broadcasting the data each comprise sending data via at least one of the group consisting of: a cellular network, a Wi-Fi network, or Bluetooth.

23. The method of claim 18, wherein the detection device comprises:
a sensor configured to sense the data corresponding to cardiac function;
a power supply configured to provide power to the sensor;
a controller powered by the power supply and configured to operate the sensor; and
communications circuitry configured to receive the data corresponding to cardiac or pulmonary function and transmit it to the data portal device.

24. The method of claim 18, wherein the server comprises learning software.
